(19) United States
(12) Patent Application Publication
(10) Pub. No.: US 2026/0223525 A1
(43) Pub. Date: Jul. 30, 2026

(54) ORGANIC COMPOUND, COMPOSITION CONTAINING SAME, ORGANIC LIGHT-EMITTING DEVICE, DISPLAY APPARATUS, IMAGING APPARATUS, ELECTRONIC EQUIPMENT, LIGHTING APPARATUS, AND MOVABLE OBJECT

(71) Applicant: CANON KABUSHIKI KAISHA, Tokyo (JP)

(72) Inventor: YUTAKA TANI, Kanagawa (JP)

(21) Appl. No.: 19/450,612

(22) Filed: Jan. 15, 2026

(30) Foreign Application Priority Data
Jan. 30, 2025 (JP) 2025-013573

(51) Int. Cl. H10K 50/11; C07D 403/04; H10K 50/16; H10K 50/844; H10K 59/131; H10K 59/80; H10K 85/30; H10K 85/60; H10K 101/10

(52) U.S. Cl. CPC H10K 50/11; C07D 403/04; H10K 50/16; H10K 50/844; H10K 59/131; H10K 59/873; H10K 85/324; H10K 85/342; H10K 85/622; H10K 85/623; H10K 85/624; H10K 85/626; H10K 85/633; H10K 85/636; H10K 85/654; H10K 85/656; H10K 85/6572; H10K 85/6574; H10K 85/6576; H10K 85/658; H10K 2101/10

(57) ABSTRACT

An organic compound represented by formula (1) or formula (2):

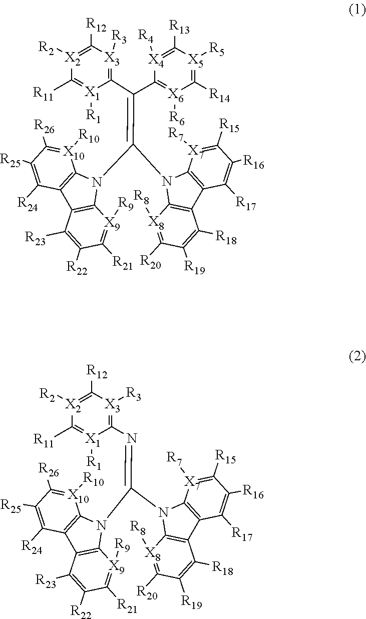

In formula (1) and formula (2), X₁ to X₁₀ are each a carbon atom or a nitrogen atom. R₁ to R₂₆ are each independently selected from the group consisting of a hydrogen atom, a deuterium atom, and substituents. R₃ and R₄ are optionally bonded together to form a ring structure.

### BACKGROUND

### Field of the Technology

The present disclosure relates to an organic compound, a composition containing the same, an organic light-emitting device, a display apparatus, an imaging apparatus, electronic equipment, a lighting apparatus, and a movable object.

### Description of the Related Art

An organic light-emitting device is an electronic device including a first electrode, a second electrode, and an organic compound layer disposed between the electrodes. The injection of electrons and holes from this pair of electrodes generates excitons of a light-emitting organic compound in the organic compound layer, and the organic light-emitting device emits light when the excitons return to the ground state. Organic light-emitting devices are also referred to as organic electroluminescence devices or organic EL devices.

Organic light-emitting devices are required to have improved characteristics such as a drive voltage, an external quantum efficiency, a color gamut, and a device lifetime, and new materials are being developed to address this disadvantage. To improve the external quantum efficiency, not only an improvement in the quantum yield of light emission from a light-emitting organic compound that is directly responsible for light emission but also high mobility of electrons and holes in an organic compound layer including a charge injection layer, a charge transport layer, a light-emitting layer, etc. is required. Furthermore, so as not to inhibit light emission from the light-emitting organic compound, it is desirable that the excited singlet level and the excited triplet level of an adjacent material be higher than those of the light-emitting organic compound. From this point of view, for example, organic compounds such as carbazole derivatives and fluorene derivatives are known as materials related to the hole transport path.

Japanese Patent Laid-Open No. 2022-150712 discloses compound A below as a compound having carbazole and discloses a light-emitting device that uses the compound and that has an improved drive lifetime. International Publication No. 2013/156125 discloses compound B below and discloses a light-emitting device that uses the compound and that has improved device lifetime and operating voltage. Japanese Patent Laid-Open No. 2011-108462 discloses compound C below as a compound having carbazole and discloses a light-emitting device that uses the compound as a hole transport layer and that has an improved device lifetime and current efficiency. International Publication No. 2015/104045 discloses compound D below and discloses a light-emitting device that uses the compound and that has improved light emission efficiency and device lifetime.

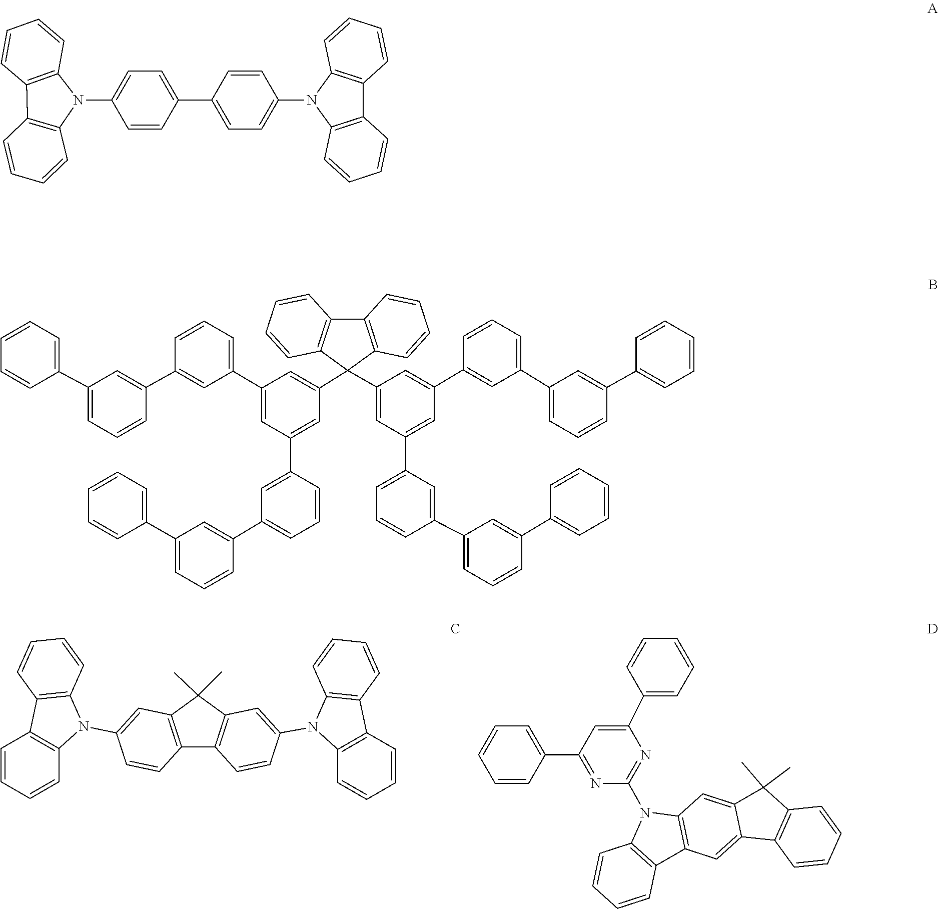

However, the present inventors have conducted extensive research and have found that there is room for improvement in the light emission efficiency in devices that use the above compounds.

### SUMMARY

The present disclosure is directed to providing an organic compound that exhibits high light emission efficiency when used in an organic light-emitting device.

The present disclosure provides an organic compound represented by formula (1) or formula (2).

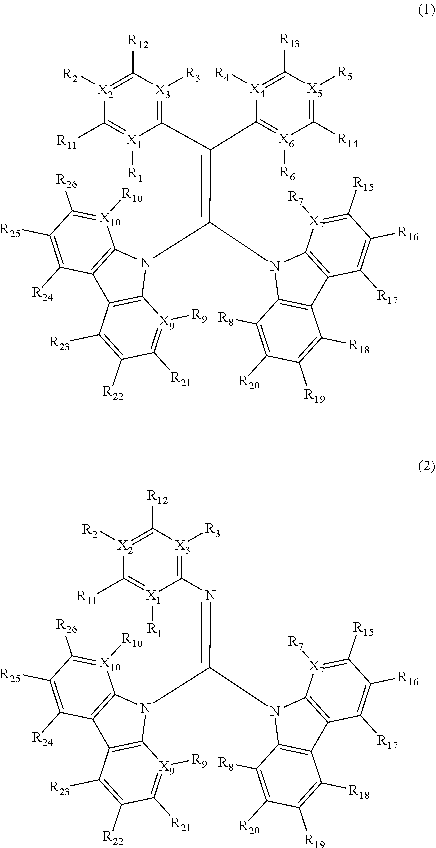

In formula (1) and formula (2), X₁ to X₁₀ are each a carbon atom or a nitrogen atom.

R₁ to R₂₆ are each independently selected from the group consisting of a hydrogen atom, a deuterium atom, a halogen atom, a substituted or unsubstituted alkyl group, a substituted or unsubstituted cycloalkyl group, a substituted or unsubstituted aryl group, a substituted or unsubstituted heteroaryl group, a substituted or unsubstituted alkoxy group, a substituted or unsubstituted aryloxy group, a substituted or unsubstituted acyl group, a substituted or unsubstituted alkoxycarbonyl group, a substituted or unsubstituted amino group, a substituted or unsubstituted imino group, a substituted or unsubstituted silyl group, a substituted or unsubstituted sulfanyl group, a substituted or unsubstituted sulfinyl group, a substituted or unsubstituted sulfonyl group, and a cyano group. R₃ and R₄ are optionally bonded together to form a ring structure.

Features of the present disclosure will become apparent from the following description of embodiments with reference to the attached drawings. The following description of embodiments is described by way of example.

### BRIEF DESCRIPTION OF THE DRAWINGS

FIG. 1A is a schematic sectional view illustrating an example of a pixel of a display apparatus according to an embodiment of the present disclosure. FIG. 1B is a schematic sectional view illustrating an example of a display apparatus using an organic light-emitting device according to an embodiment of the present disclosure.

FIG. 2 is a schematic view illustrating an example of a display apparatus using an organic light-emitting device according to an embodiment of the present disclosure.

FIG. 3A is a schematic view illustrating an example of an imaging apparatus according to an embodiment of the present disclosure. FIG. 3B is a schematic view illustrating an example of electronic equipment according to an embodiment of the present disclosure.

FIG. 4A is a schematic view illustrating an example of a display apparatus according to an embodiment of the present disclosure. FIG. 4B is a schematic view illustrating an example of a foldable display apparatus.

FIG. 5A is a schematic view illustrating an example of a lighting apparatus according to an embodiment of the present disclosure. FIG. 5B is a schematic view illustrating an automobile, which is an example of a movable object according to an embodiment of the present disclosure.

FIG. 6A is a schematic view illustrating an example of a wearable device according to an embodiment of the present disclosure. FIG. 6B is a schematic view illustrating an example of the wearable device according to an embodiment of the present disclosure, the wearable device including an imaging apparatus.

### DESCRIPTION OF THE EMBODIMENTS

Embodiments of the present disclosure will be described below. The present disclosure is not limited to the description below, and a person skilled in the art can easily understand that various changes in form and detail can be made without departing from the gist and the scope of the present disclosure. That is, the present disclosure is not construed as being limited by the following description. Characteristic values are values at 25° C. unless otherwise specified.

### Organic Compound Represented by Formula (1) or (2)

As a result of studies, the inventors have found an organic compound that has improved light emission efficiency and that can be used for a hole injection/transport layer and a light-emitting layer of an organic light-emitting device, the organic compound being represented by a formula (1) or (2) below.

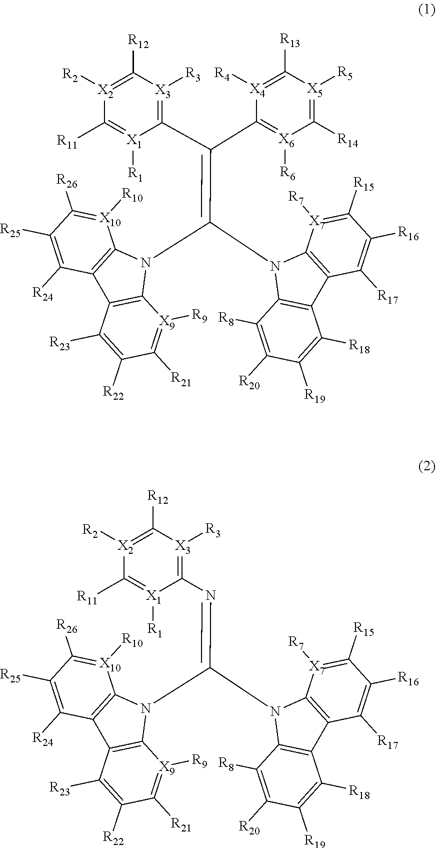

In the formulas (1) and (2), X₁ to X₁₀ are each a carbon atom or a nitrogen atom.

R₁ to R₂₆ are each independently selected from the group consisting of a hydrogen atom, a deuterium atom, a halogen atom, a substituted or unsubstituted alkyl group, a substituted or unsubstituted cycloalkyl group, a substituted or unsubstituted aryl group, a substituted or unsubstituted heteroaryl group, a substituted or unsubstituted alkoxy group, a substituted or unsubstituted aryloxy group, a substituted or unsubstituted acyl group, a substituted or unsubstituted alkoxycarbonyl group, a substituted or unsubstituted amino group, a substituted or unsubstituted imino group, a substituted or unsubstituted silyl group, a substituted or unsubstituted sulfanyl group, a substituted or unsubstituted sulfinyl group, a substituted or unsubstituted sulfonyl group, and a cyano group. R₃ and R₄ may be bonded together to form a ring structure.

In this specification, the halogen atom in R₁ to R₂₆ is any of a fluorine atom, a chlorine atom, a bromine atom, and an iodine atom. Of these, a fluorine atom is preferred in view of thermal stability.

In this specification, the alkyl group in R₁ to R₂₆ may be any of a linear alkyl group and a branched alkyl group. The linear alkyl group may have 1 to 20 carbon atoms or may have 1 to 8 carbon atoms. The branched alkyl group may have 3 to 20 carbon atoms or may have 3 to 8 carbon atoms.

In this specification, the cycloalkyl group in R₁ to R₂₆ may have 3 to 20 carbon atoms or may have 3 to 10 carbon atoms. A cyclohexyl group or a cyclopentyl group is preferred. Some of carbon atoms of the cycloalkyl group may be replaced with an oxygen atom, however, it is not preferable to replace two consecutive carbon atoms with oxygen atoms. One carbon atom may be replaced with an oxygen atom.

In this specification, the aryl group in R₁ to R₂₆ may be an aryl group having 6 to 60 carbon atoms. Specific examples of the aryl group include a phenyl group, a naphthyl group, a biphenyl group, a fluorenyl group, a phenanthryl group, an anthryl group, a pyrenyl group, a chrysenyl group, a pyridyl group, a pyrimidyl group, a triazinyl group, a carbazolyl group, a dibenzofuryl group, and a dibenzothienyl group.

In this specification, the heteroaryl group in R₁ to R₂₆ may be a heteroaryl group having 3 to 59 carbon atoms. The heteroatom may be an oxygen atom, a sulfur atom, a phosphorus atom, a selenium atom, or a tellurium atom. Specific examples of the heteroaryl group include a pyridyl group, a pyrimidyl group, a triazinyl group, a carbazolyl group, a dibenzofuryl group, and a dibenzothienyl group.

In this specification, the alkyl group in the alkoxy group in R₁ to R₂₆ may be any of a linear alkyl group, a branched alkyl group, and a cycloalkyl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms.

In this specification, the aryl group in the aryloxy group in R₁ to R₂₆ may be an aryl group having 6 to 60 carbon atoms. Specific examples of the aryl group include a phenyl group, a naphthyl group, a biphenyl group, a fluorenyl group, a phenanthryl group, an anthryl group, a pyrenyl group, and a chrysenyl group. A heteroaryl group may be included in the aryl group in the aryloxy group. Examples of the heteroaryl group include a pyridyl group, a pyrimidyl group, a triazinyl group, a carbazolyl group, a dibenzofuryl group, and a dibenzothienyl group.

In this specification, the alkyl group in the acyl group in R₁ to R₂₆ may be any of a linear alkyl group, a branched alkyl group, and a cycloalkyl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms.

In this specification, the alkyl group in the alkoxycarbonyl group in R₁ to R₂₆ may be any of a linear alkyl group, a branched alkyl group, and a cycloalkyl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms.

In this specification, the amino group in R₁ to R₂₆ may be any of an unsubstituted amino group, a monosubstituted amino group, and a disubstituted amino group. The substituent may be any of a linear alkyl group, a branched alkyl group, a cycloalkyl group, and an aryl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms. The aryl group may be an aryl group having 6 to 60 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. The heteroaryl group may be a heteroaryl group having 3 to 59 carbon atoms. Specifically, the heteroaryl group may be a pyridyl group, a pyrimidyl group, a triazinyl group, a carbazolyl group, a dibenzofuryl group, a dibenzothienyl group, or the like.

In this specification, the imino group in R₁ to R₂₆ may be any of an unsubstituted imino group and a substituted imino group. The substituent may be any of a linear alkyl group, a branched alkyl group, a cycloalkyl group, an aryl group, and a heteroaryl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms. The aryl group may be an aryl group having 6 to 60 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. The heteroaryl group may be a heteroaryl group having 3 to 59 carbon atoms. Specifically, the heteroaryl group may be a pyridyl group, a pyrimidyl group, a triazinyl group, a carbazolyl group, a dibenzofuryl group, a dibenzothienyl group, or the like.

In this specification, the silyl group in R₁ to R₂₆ may be any of a trialkylsilyl group, a dialkylarylsilyl group, an alkyldiarylsilyl group, and a triarylsilyl group. The alkyl groups may each be an alkyl group having an independent number of carbon atoms. The alkyl group may be any of a linear alkyl group, a branched alkyl group, and a cycloalkyl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms. The aryl groups and the heteroaryl groups may be of different types.

The aryl group may be an aryl group having 6 to 60 carbon atoms, and the heteroaryl group may be a heteroaryl group having 3 to 59 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. Specifically, the heteroaryl group may be a pyridyl group, a pyrimidyl group, a triazinyl group, a carbazolyl group, a dibenzofuryl group, a dibenzothienyl group, or the like.

In this specification, the sulfanyl group in R₁ to R₂₆ may be any of an unsubstituted sulfanyl group and a substituted sulfanyl group. The substituent may be any of a linear alkyl group, a branched alkyl group, a cycloalkyl group, and an aryl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms. The aryl group may be an aryl group having 6 to 60 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. The heteroaryl group may be a heteroaryl group having 3 to 59 carbon atoms. Specifically, the heteroaryl group may be a pyridyl group, a pyrimidyl group, a triazinyl group, a carbazolyl group, a dibenzofuryl group, a dibenzothienyl group, or the like.

In this specification, the substituent in the sulfinyl group in R₁ to R₂₆ may be any of a linear alkyl group, a branched alkyl group, a cycloalkyl group, and an aryl group. The linear alkyl group may have 1 to 20 carbon atoms or may have 1 to 8 carbon atoms. The branched alkyl group may have 3 to 20 carbon atoms or may have 3 to 8 carbon atoms. The cycloalkyl group may have 3 to 20 carbon atoms or may have 3 to 10 carbon atoms. The aryl group may be an aryl group having 6 to 60 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. The heteroaryl group may be a heteroaryl group having 3 to 59 carbon atoms. Specifically, the heteroaryl group may be a pyridyl group, a pyrimidyl group, a triazinyl group, a carbazolyl group, a dibenzofuryl group, a dibenzothienyl group, or the like.

In this specification, the substituent in the sulfonyl group in R₁ to R₂₆ may be any of a linear alkyl group, a branched alkyl group, a cycloalkyl group, and an aryl group. The linear alkyl group may have 1 to 20 carbon atoms or may have 1 to 8 carbon atoms. The branched alkyl group may have 3 to 20 carbon atoms or may have 3 to 8 carbon atoms.

The cycloalkyl group may have 3 to 20 carbon atoms or may have 3 to 10 carbon atoms. The aryl group may be an aryl group having 6 to 60 carbon atoms. Specifically, the aryl group may be a phenyl group, a naphthyl group, a biphenyl group, a fluorenyl group, a phenanthryl group, an anthryl group, a pyrenyl group, a chrysenyl group, or the like. The heteroaryl group may be a heteroaryl group having 3 to 59 carbon atoms. Specifically, the heteroaryl group may be a pyridyl group, a pyrimidyl group, a triazinyl group, a carbazolyl group, a dibenzofuryl group, a dibenzothienyl group, or the like.

In this specification, R₃ and R₄ may be bonded together to form a ring structure. The expression “forming a ring structure” refers to forming a ring structure by bonding between R₃ and R₄ with or without a divalent cross-linking group. The divalent cross-linking group may be any of an alkylene group, an alkylidene group, an alkenylene group, an alkynylene group, a carbonyl group, an ether group, a sulfanyl group, a sulfinyl group, a sulfonyl group, an imino group, and a silylene group. The alkylene group may be specifically a methylene group, a 1,1-ethylene group, a 1,2-ethylene group, a phenylmethylene group, a 9,9-fluorenylene group, or the like but is not limited thereto. The alkylidene group may be specifically an ethylidene group, an isopropylidene group, or the like but is not limited thereto. The alkenylene group may be specifically a 1,2-vinylene group, a 1,2-propylene group, or the like but is not limited thereto. The alkynylene group may be specifically an ethynylene group. The imino group may be specifically a methylimino group, an ethylimino group, a phenylimino group, or the like but is not limited thereto. The silylene group may be specifically a dimethylsilylene group, a diphenylsilylene group, or the like but is not limited thereto. In the case where a divalent cross-linking group is not used, R₃ and R₄ are bonded to each other to form a five-membered ring.

The alkyl group or the cycloalkyl group in this specification may further have a substituent, and the substituent may be a halogen atom, a cyano group, or a nitro group. The halogen atom with which the alkyl group can be substituted is any of a fluorine atom, a chlorine atom, a bromine atom, and an iodine atom. Of these, a fluorine atom is preferred in view of thermal stability. In the alkyl group or the cycloalkyl group, one methylene group or two or more methylene groups that are not adjacent to each other can be substituted with an —O— group, a —S— group, a —C(═O)— group, a —C(═O)O— group, an —O(C═O)— group, a —CH═CH— group, or a —C≡C— group, and a hydrogen atom can be substituted with a fluorine atom.

The aryl group in this specification may have an alkyl group as a substituent. In the alkyl group, one methylene group or two or more methylene groups that are not adjacent to each other can be substituted with an —O— group, a —S— group, a —C(═O)— group, a —C(═O)O— group, an —O(C═O)— group, a —CH═CH— group, or a —C≡C— group, and a hydrogen atom can be replaced with a fluorine atom.

Specific examples of the aryl group in this specification, the examples including those further having a substituent, include, but are not limited to, a phenyl group, a 1-naphthyl group, a 2-naphthyl group, a 4-biphenyl group, a 2-fluorenyl group, a 9-phenanthryl group, a 2-anthryl group, a 1-pyrenyl group, a 1-imidazolyl group, a 2-furyl group, a 3-benzofuryl group, a 4-dibenzofuryl group, a 2-thienyl group, a 3-benzothienyl group, a 2-dibenzothienyl group, a 2-pyridyl group, a 2-pyrimidinyl group, a 1-indolyl group, a 2-indolyl group, a 9-carbazolyl group, a p-chlorophenyl group, an o-tolyl group, a 4-methoxyphenyl group, a 4′-(1-hexynyl) phenyl group, a 2-(1-(1,1,2,2,3,3,4,4,5,5,6,6,7,7,8,8-hexadecafluoro)octyloxycarbonyl)phenyl group, a 4′-cyanobiphenyl group, a 2-(9,9-dimethyl)fluorenyl group, and a 3-(9,9-dioctyl)fluorenyl group.

R₃ and R₄ in the formula (1) may be bonded together to form a ring structure. When R₃ and R₄ are bonded together to form a ring structure, R₃ and R₄ may be directly bonded to each other to form a five-membered ring. R₃ and R₄ may form any of a methylene group, an ethylene group, an ether group, a thioether group, a sulfone group, a sulfoxide group, an imino group, and a carbonyl group that may have a substituent.

When R₃ and R₄ are bonded together to form a ring structure, R₁, R₂, and R₅ to R₂₆ may be any of a hydrogen atom, a deuterium atom, the alkyl group, the aryl group, the heteroaryl group, the silyl group, and the cyano group.

Specific examples of the structural formula of the organic compound according to the present disclosure are illustrated below. In the following exemplary compounds, Me represents a methyl group, Pr represents a propyl group, Bu represents a butyl group, Ph represents a phenyl group, Hex represents a hexyl group, Mes represents a mesityl group, i.e., a 2,4,6-trimethylphenyl group, and TIPS represents a triisopropylsilyl group. The prefixes n, i, and t of the abbreviated substituents represent normal, iso, and tertiary, respectively. That is, tBu represents a tertiary butyl group.

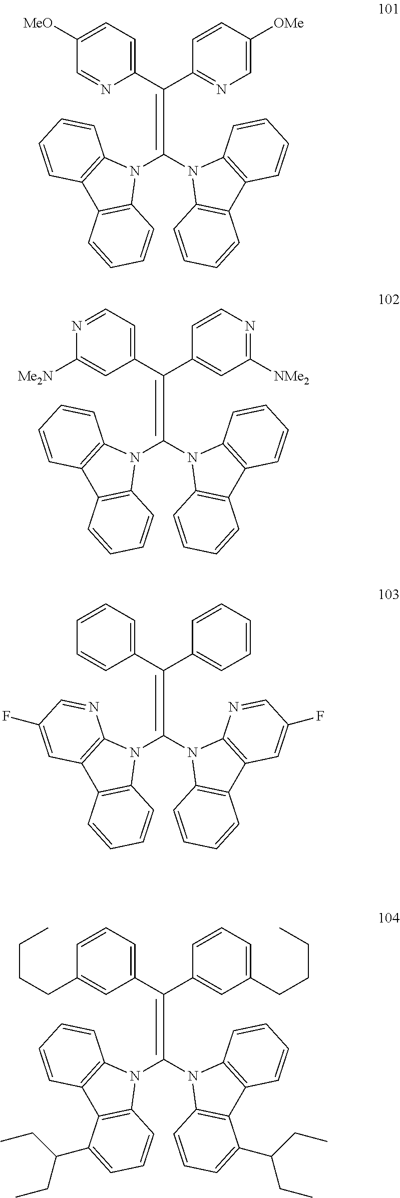

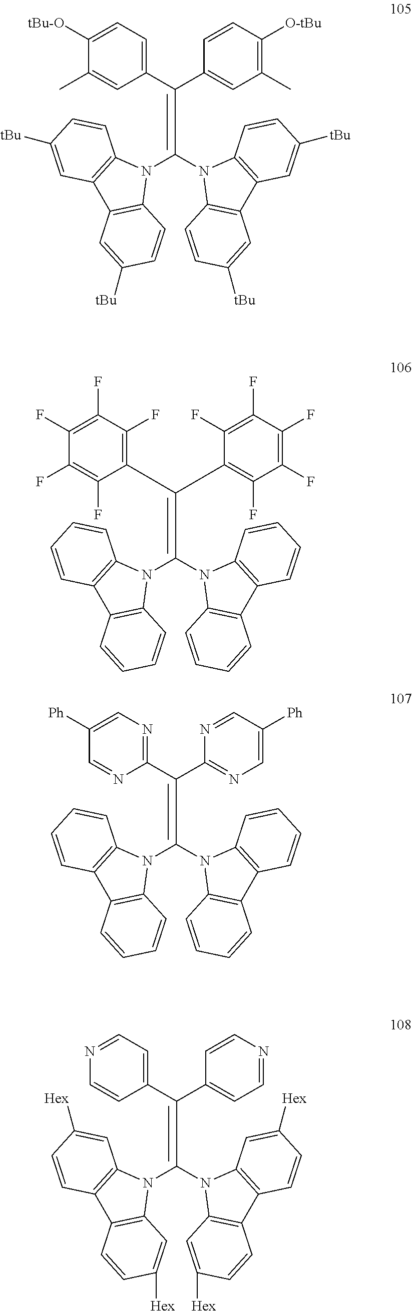

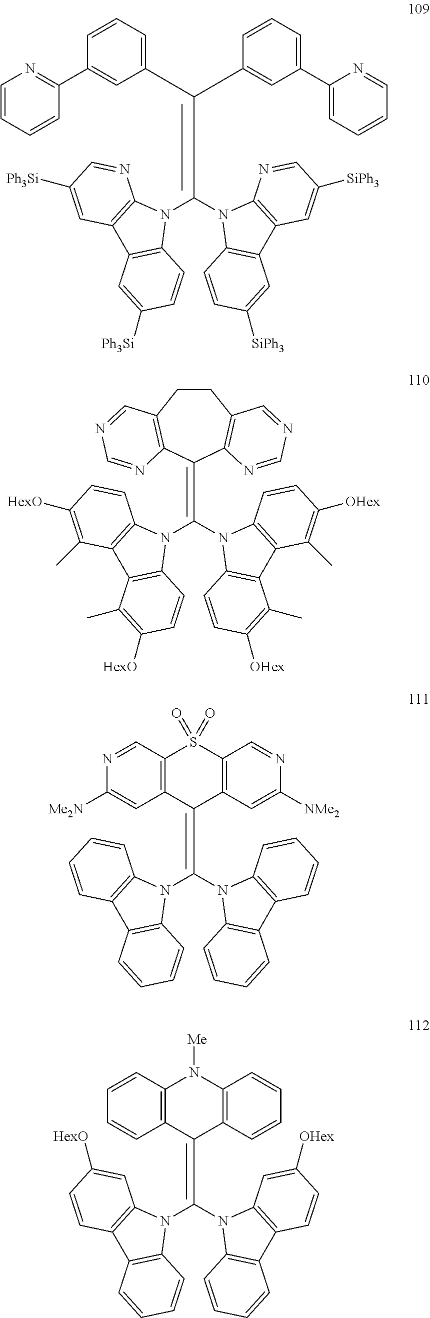

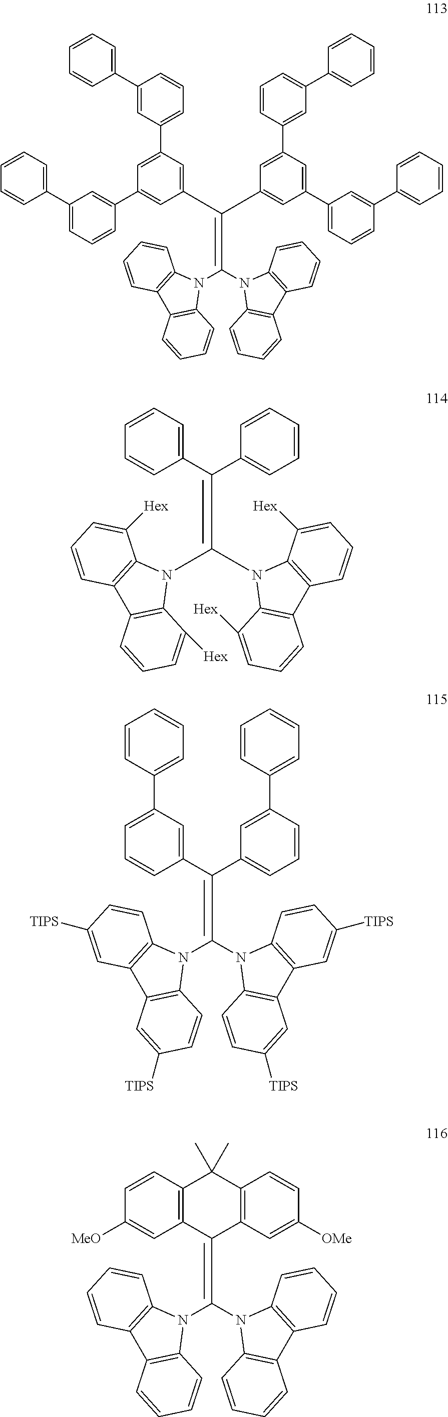

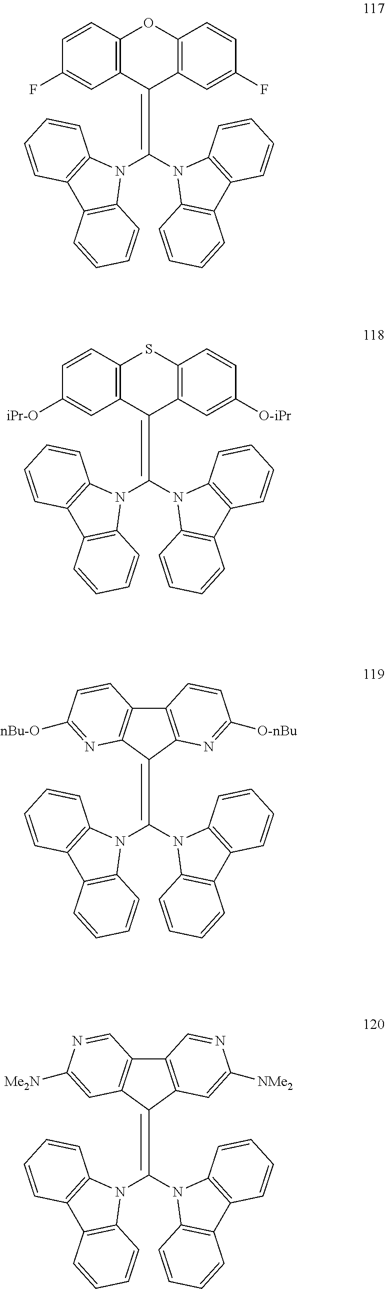

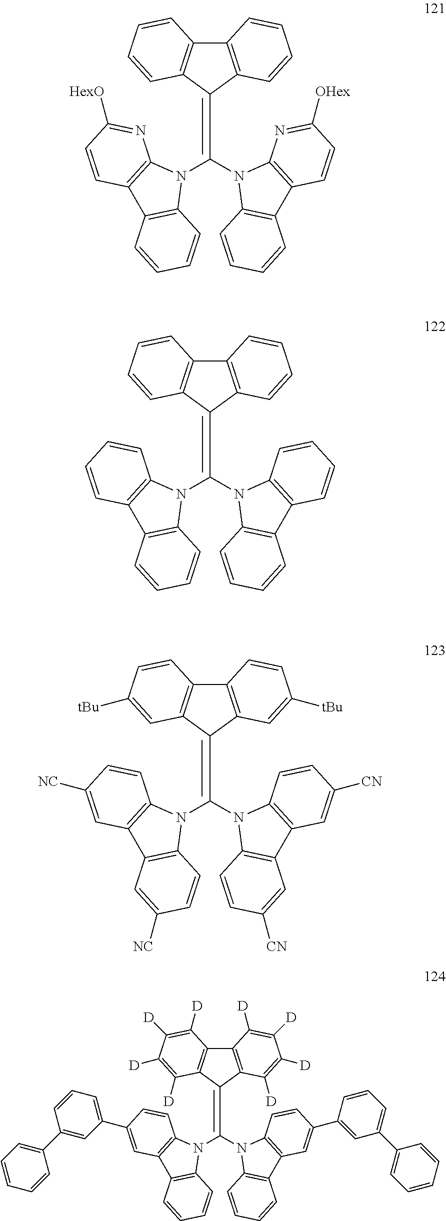

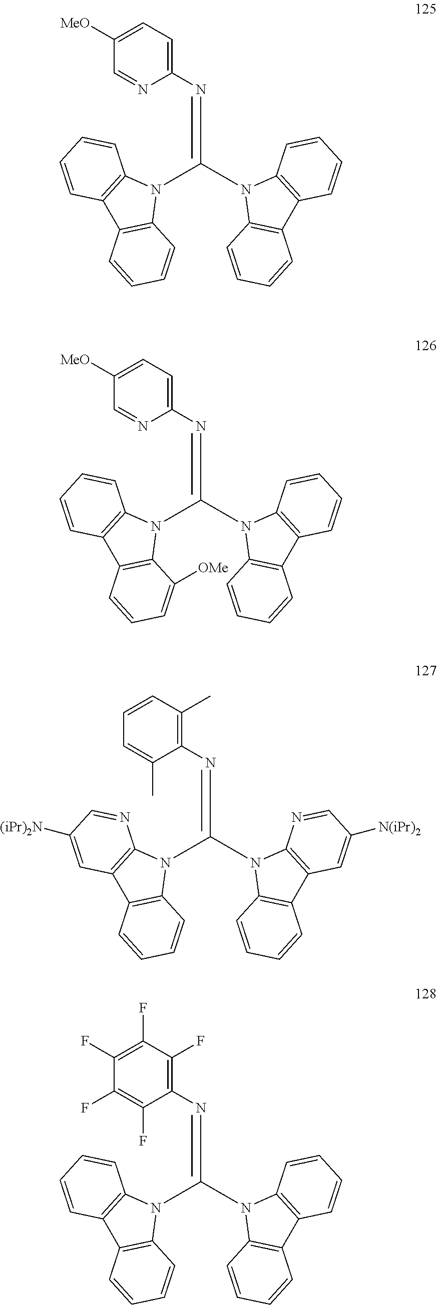

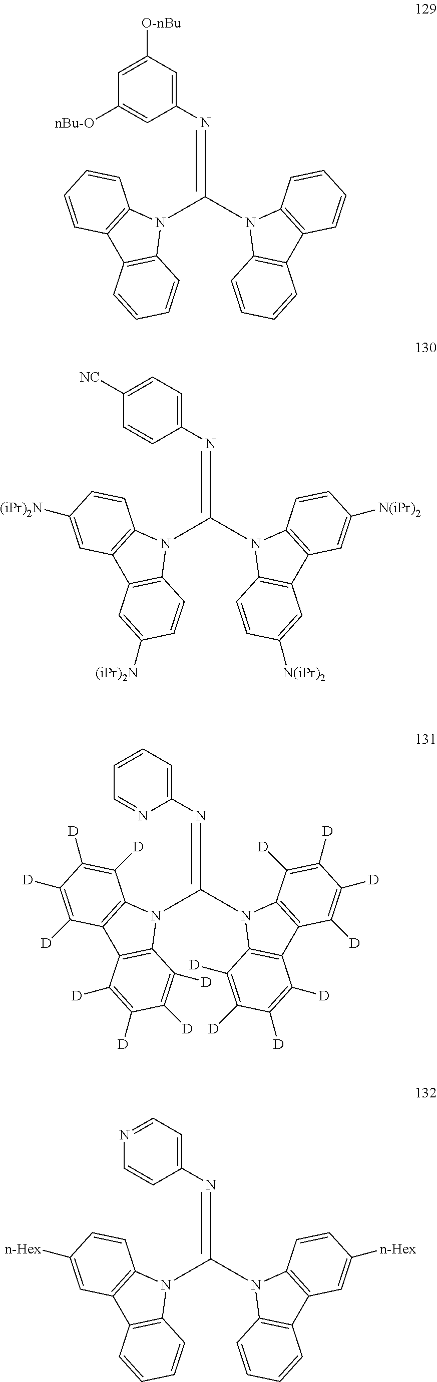

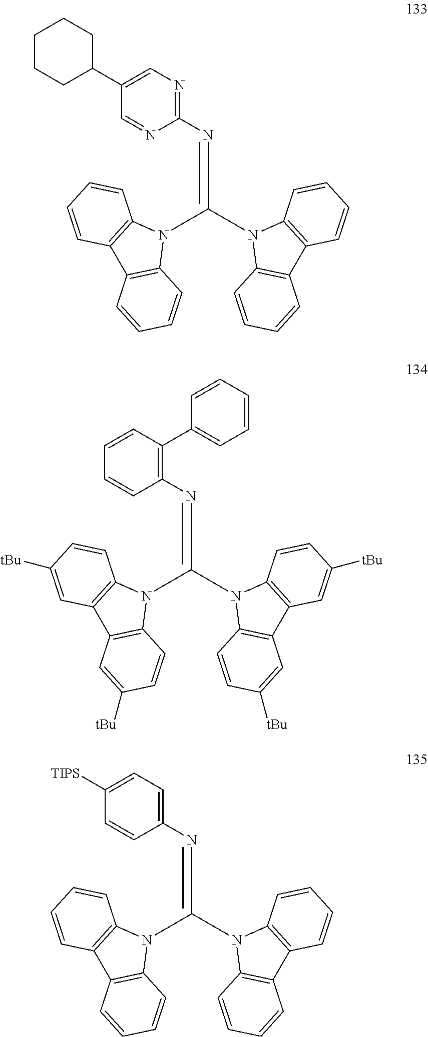

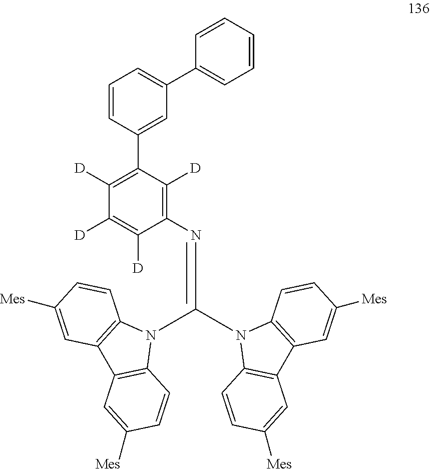

### Organic Compound Layer Included in Organic Light-Emitting Device According to Embodiment of Present Disclosure

Next, an organic compound layer included in an organic light-emitting device according to an embodiment of the present disclosure will be described.

An organic light-emitting device according to the present embodiment includes a first electrode and a second electrode, which are a pair of electrodes, and an organic compound layer disposed between the electrodes. In the organic light-emitting device of the present embodiment, the organic compound layer may be a single layer or a laminate of a plurality of layers as long as the organic compound layer includes a light-emitting layer. The pair of electrodes may be an anode and a cathode.

When the organic compound layer is a laminate of a plurality of layers, the organic compound layer may include a light-emitting layer. The organic compound layer may include, besides the light-emitting layer, for example, a hole injection layer, a hole transport layer, an electron-blocking layer, a hole/exciton-blocking layer, an electron transport layer, and an electron injection layer.

The light-emitting layer may be a single layer or a laminate of a plurality of layers. When the light-emitting layer is composed of a plurality of light-emitting layers, a charge generation layer may be disposed between the light-emitting layers. The charge generation layer may be composed of a compound having a lower LUMO energy than the hole transport layer. That is, the LUMO energy of the charge generation layer may be lower than the HOMO energy of the hole transport layer. Here, the molecular orbital energy of the organic compound layer may be the molecular orbital energy of the organic compound having the highest weight ratio in the organic compound layer.

Here, the HOMO energy level and the LUMO energy level are described as “higher” as they are closer to the vacuum level. The expression “the LUMO energy of the charge generation layer is lower than the HOMO energy of the hole transport layer” indicates that the LUMO energy of the charge generation layer is farther from the vacuum level than the HOMO energy of the hole transport layer.

HOMO and LUMO in this specification can be calculated by molecular orbital calculation. The molecular orbital calculation may be performed according to the density functional theory (DFT) or the like using B3LYP as the functional and 6-31G* as the basis function, for example. The molecular orbital calculation can be performed by using, for example, Gaussian 09 (Gaussian 09, Revision C.01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford CT, 2010.).

HOMO and LUMO in this specification can be calculated using ionization potentials and band gaps. HOMO can be estimated by measuring the ionization potential. The ionization potential can be measured by dissolving a compound to be measured in a solvent, such as toluene, or vapor-depositing a compound to be measured on a substrate, such as a glass substrate, and then performing measurement with a measurement apparatus, such as AC-3. The band gap can be measured by dissolving a compound to be measured in a solvent, such as toluene, and irradiating the resulting solution with excitation light. The band gap can be measured by measuring the absorption edge of the absorption spectrum of the excitation light. Alternatively, the measurement can be performed by vapor-depositing a compound to be measured on a substrate, such as a glass substrate, and irradiating the vapor-deposited film with excitation light. The band gap can be measured by measuring the absorption edge of the absorption spectrum of the vapor-deposited film when the film absorbs the excitation light.

LUMO can be calculated using the values of the band gap and the ionization potential. LUMO can be estimated by subtracting the value of the ionization potential from the value of the band gap.

LUMO can also be estimated from a reduction potential. For example, a one-electron reduction potential is estimated by cyclic voltammetry (CV) measurement. The CV measurement can be performed, for example, in a DMF solution of 0.1 M tetrabutylammonium perchlorate using Ag/Ag⁺ as a reference electrode, Pt as a counter electrode, and glassy carbon as a working electrode. LUMO can be estimated by adding the difference between the obtained reduction potential of the compound and the reduction potential of ferrocene to −4.8 eV.

In the organic light-emitting device of the present embodiment, at least one layer of the organic compound layer contains the organic compound according to the present embodiment. Specifically, the organic compound according to the present embodiment is contained in any of the hole injection layer, the hole transport layer, the electron-blocking layer, the light-emitting layer, the hole/exciton-blocking layer, the electron transport layer, the electron injection layer, and the like and is preferably contained in the hole injection layer, the hole transport layer, the electron-blocking layer, or the light-emitting layer. The transport layers between the first electrode and the light-emitting layer can be collectively referred to as a first charge transport layer. The transport layers between the second electrode and the light-emitting layer can be collectively referred to as a second charge transport layer. That is, the light-emitting layer is in contact with the first charge transport layer and is in contact with the second charge transport layer.

In the organic light-emitting device of the present embodiment, when the organic compound according to the present embodiment is contained in a light-emitting layer, the light-emitting layer may be a layer containing, in addition to the organic compound according to the present embodiment, a first organic compound or a second organic compound different from the first organic compound. When the light-emitting layer contains the second organic compound, the light-emitting layer may be a layer that further contains a third organic compound different from the second organic compound. The first organic compound may have a lower lowest excited singlet energy than the lowest excited singlet energy of the organic compound according to the present disclosure. The second organic compound may have a lower lowest excited triplet energy than the lowest excited triplet energy of the organic compound according to the present disclosure. The third organic compound may have a lowest excited triplet energy lower than the lowest excited triplet energy of the organic compound according to the present disclosure and higher than the lowest excited triplet energy of the second organic compound. Here, when the light-emitting layer is a layer containing the first organic compound or the second organic compound, the first organic compound may be a fluorescent dopant of the light-emitting layer. The second organic compound may be a phosphorescent dopant of the light-emitting layer. The third organic compound may be an assist material. The organic compound according to the present disclosure may be a host material of the light-emitting layer.

Here, the host is a compound having the highest weight ratio among the compounds constituting the light-emitting layer.

The dopant is a compound that has a lower weight ratio than the host among the compounds constituting the light-emitting layer and that is responsible for main light emission. The assist material is a compound that has a lower weight ratio than the host among the compounds constituting the light-emitting layer and that assists the light emission of the dopant. The assist material is also referred to as a second host.

When the organic compound according to the present embodiment is used as a host in the light-emitting layer, the concentration of the host is preferably 80% by weight or more and 99.99% by weight or less, more preferably 90% by weight or more and 99.9% by weight or less relative to the entire light-emitting layer. The entire light-emitting layer refers to the total weight of the compounds constituting the light-emitting layer.

The lowest excited triplet energy of the first charge transport layer is preferably larger than the lowest excited triplet energy of the first organic compound. The lowest excited triplet energy of the second charge transport layer is preferably larger than the lowest excited triplet energy of the first organic compound. The lowest excited triplet energy of each charge transport layer can be estimated by the lowest excited triplet energy of the constituent material of the layer. When the charge transport layer is composed of a plurality of materials, the lowest excited triplet energy may be the lowest excited triplet energy of a compound having a high weight ratio.

The inventors have conducted various studies and have found that when the organic compound according to the present embodiment is used as a host of a light-emitting layer, light output with high efficiency and high luminance is exhibited. This light-emitting layer may be composed of a single layer or multiple layers, can contain another host or a light-emitting assist material in combination, and can also contain a light-emitting material having another emission color to thereby form a color mixture of the emission color of the present embodiment and the other emission color. The term “multiple layers” means a state in which a plurality of light-emitting layers are stacked. In this case, the emission color of the organic light-emitting device is not limited to the same hue as the emission color of the single layer. More specifically, the emission color may be white or an intermediate color. In the case of white, red light, blue light, and green light may be emitted from the light-emitting layers to obtain white light, or complementary emission colors may be combined to obtain white light.

The organic compound according to the present embodiment can be used as a constituent material of an organic compound layer other than the light-emitting layer constituting the organic light-emitting device of the present embodiment. Specifically, the organic compound according to the present embodiment may be used as a constituent material of the electron transport layer, the electron injection layer, the hole transport layer, the hole injection layer, the hole-blocking layer, or the like.

In manufacturing the organic light-emitting device according to the present embodiment, for example, a known low-molecular-weight or high-molecular-weight hole injection compound or hole transport compound, another compound serving as the host, a light-emitting organic compound, an electron injection compound, or an electron transport compound can be used in combination as necessary. Examples of these compounds will be described below.

The hole injection/transport material is preferably a material having a high hole mobility so as to facilitate hole injection from the anode and to enable the injected holes to be transported to the light-emitting layer. The hole injection/transport material is preferably a material having a high glass transition temperature in order to reduce degradation of the film quality, such as crystallization, in the organic light-emitting device. Examples of the low-molecular-weight and high-molecular-weight materials having such hole injection/transport performance include triarylamine derivatives, arylcarbazole derivatives, phenylenediamine derivatives, triazole derivatives, oxadiazole derivatives, imidazole derivatives, stilbene derivatives, phthalocyanine derivatives, porphyrin derivatives, polyarylamine derivatives, polyvinylcarbazole derivatives, polythiophene derivatives, and conductive polymers such as PEDOT-PSS, copolymers thereof, and mixtures thereof. Furthermore, the above hole injection/transport materials are also suitable for use in an electron-blocking layer.

Specific examples of the compound used as the hole injection/transport material are shown below, but of course, the hole injection/transport material is not limited thereto.

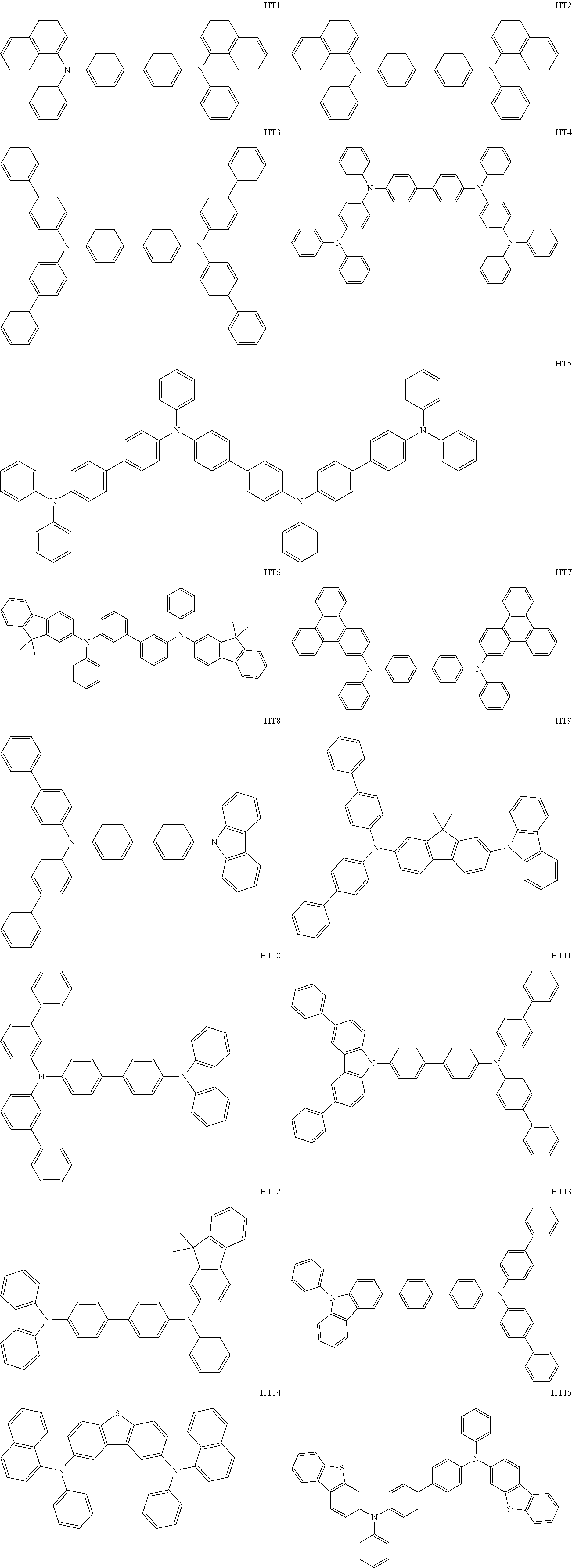

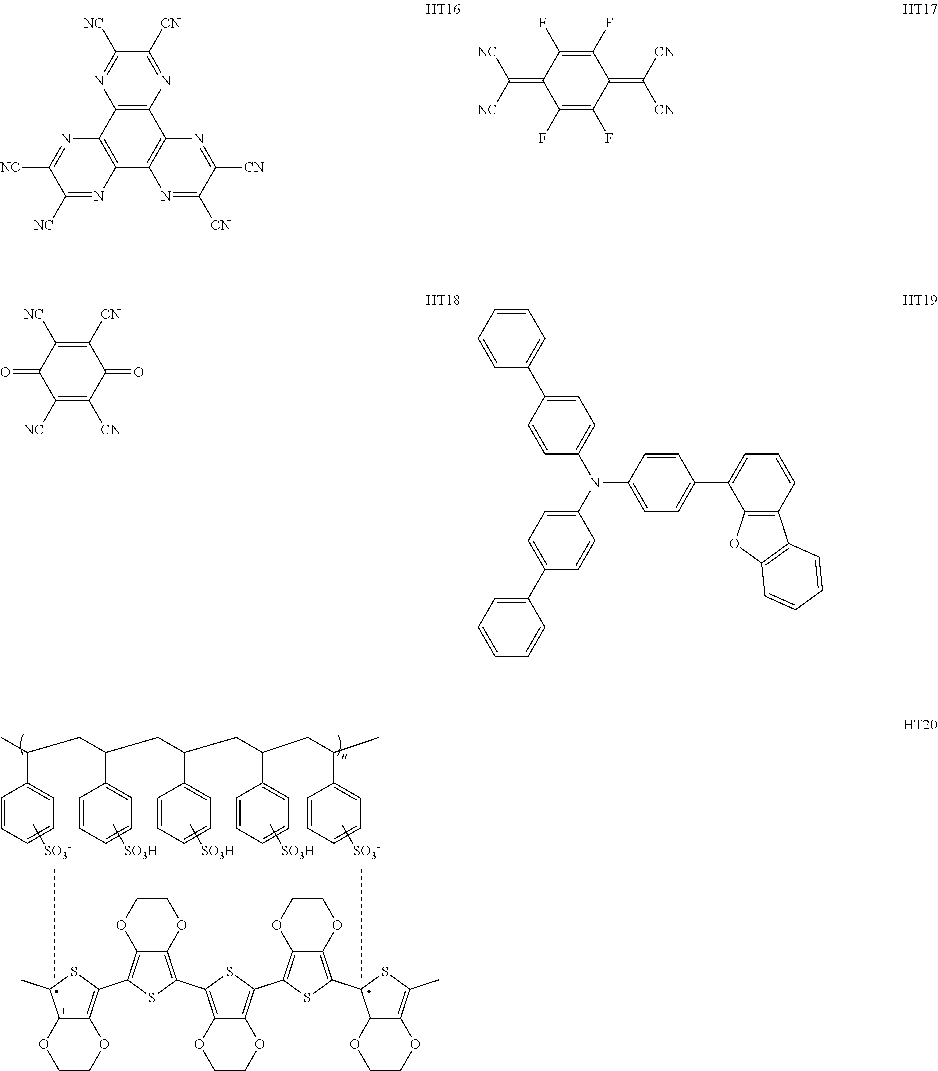

Among the hole transport materials shown above, HT16 to HT18 can be used in the layer in contact with the anode to reduce the drive voltage. HT16 is widely used in organic light-emitting devices. HT2, HT3, HT4, HT5, HT6, HT10, and HT12 may be used in an organic compound layer adjacent to HT16. A plurality of materials may be used in a single organic compound layer.

Examples of the light-emitting material that is mainly related to the light-emitting function include fused-ring compounds (such as fluorene derivatives, naphthalene derivatives, pyrene derivatives, perylene derivatives, tetracene derivatives, anthracene derivatives, and rubrene), quinacridone derivatives, coumarin derivatives, stilbene derivatives, organoaluminum complexes such as tris(8-quinolinolato)aluminum, iridium complexes, platinum complexes, rhenium complexes, copper complexes, europium complexes, ruthenium complexes, and polymer derivatives such as poly(phenylene vinylene) derivatives, poly(fluorene) derivatives, and poly(phenylene) derivatives.

Specific examples of the compound used as the light-emitting material are shown below, but of course, the light-emitting material is not limited thereto.

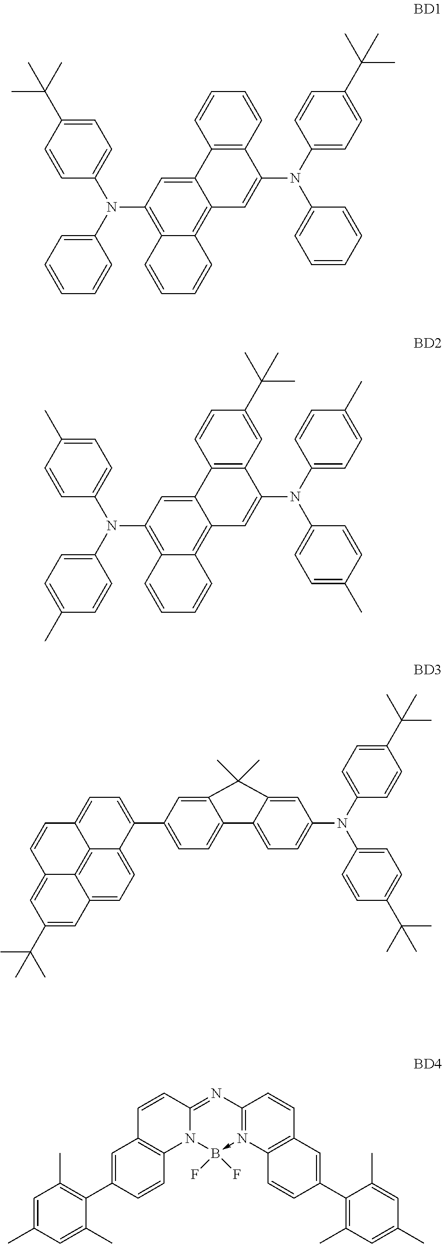

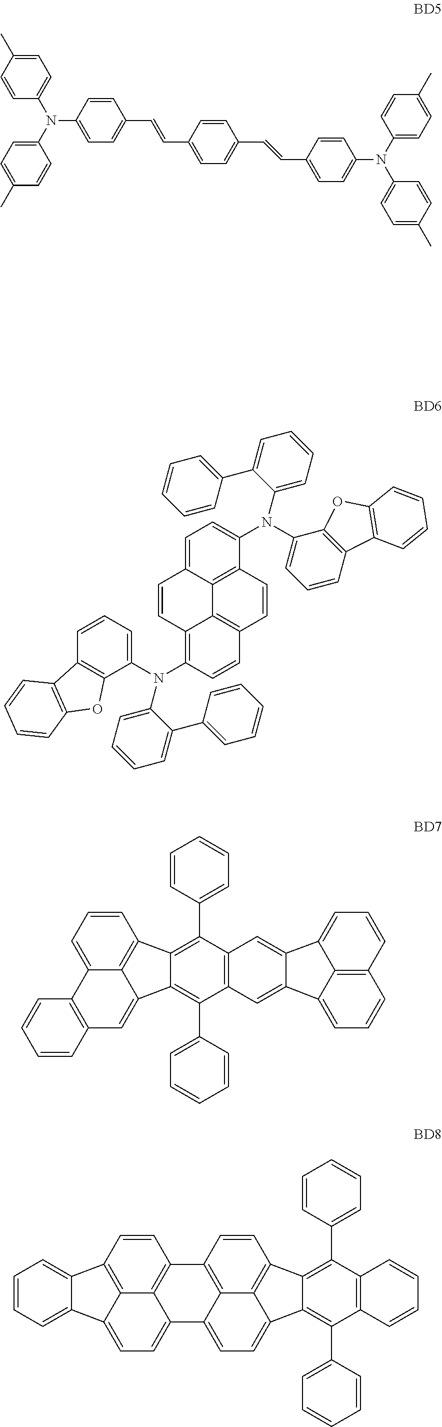

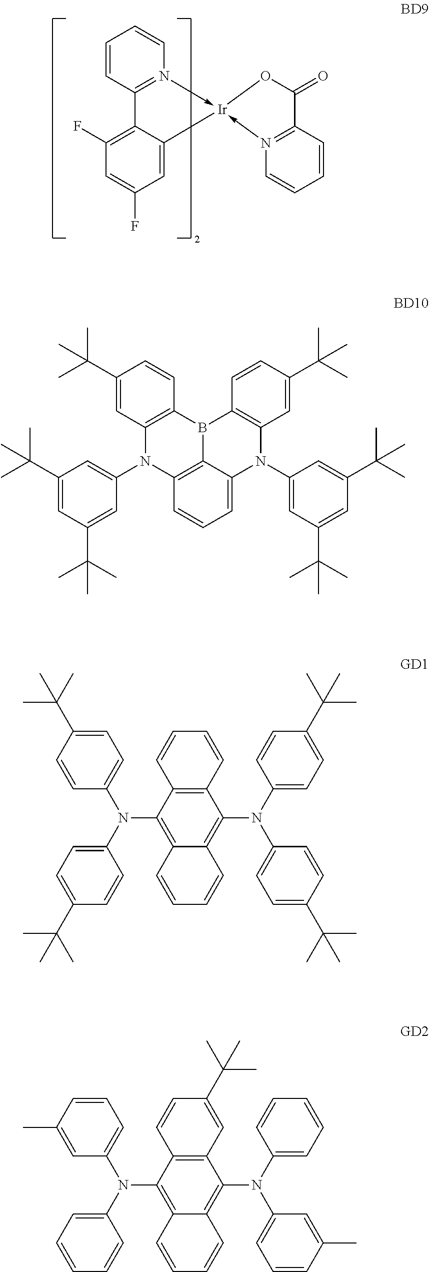

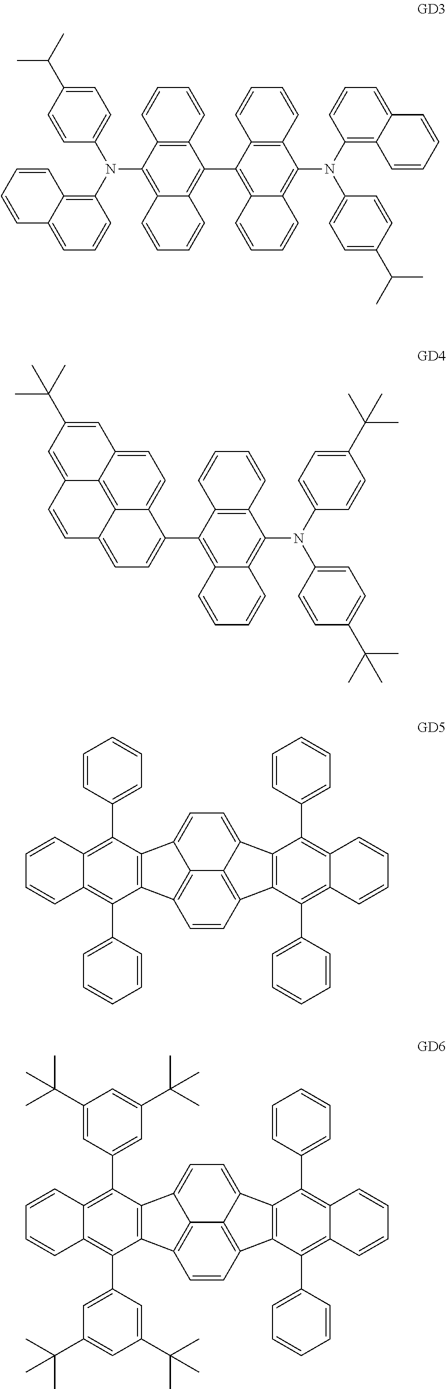

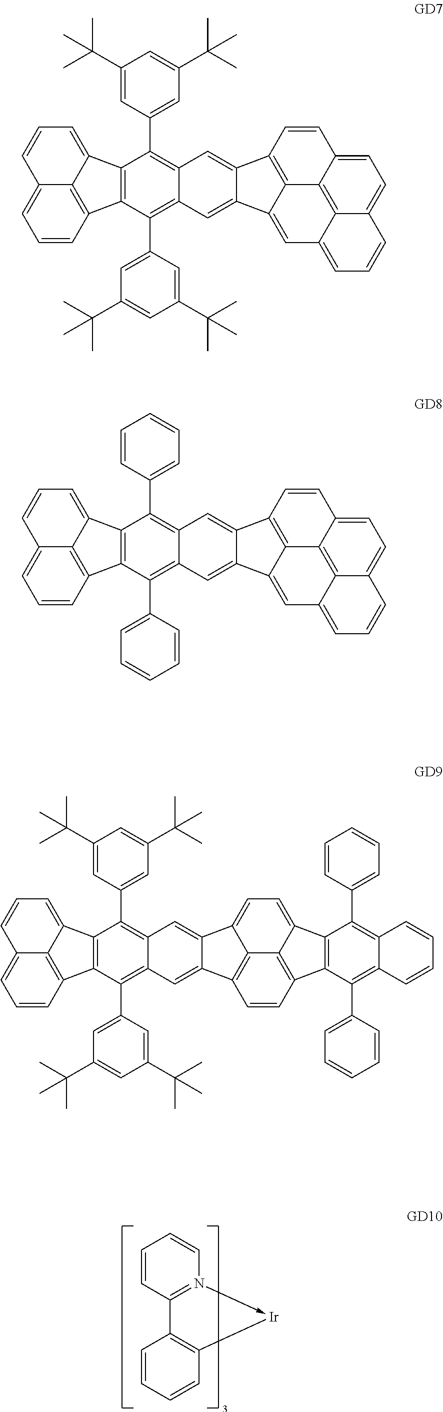

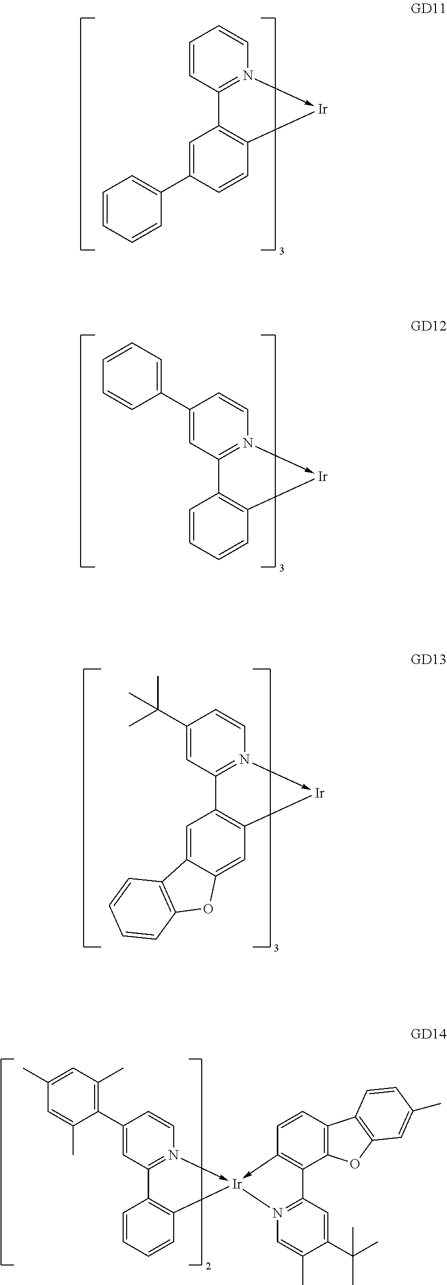

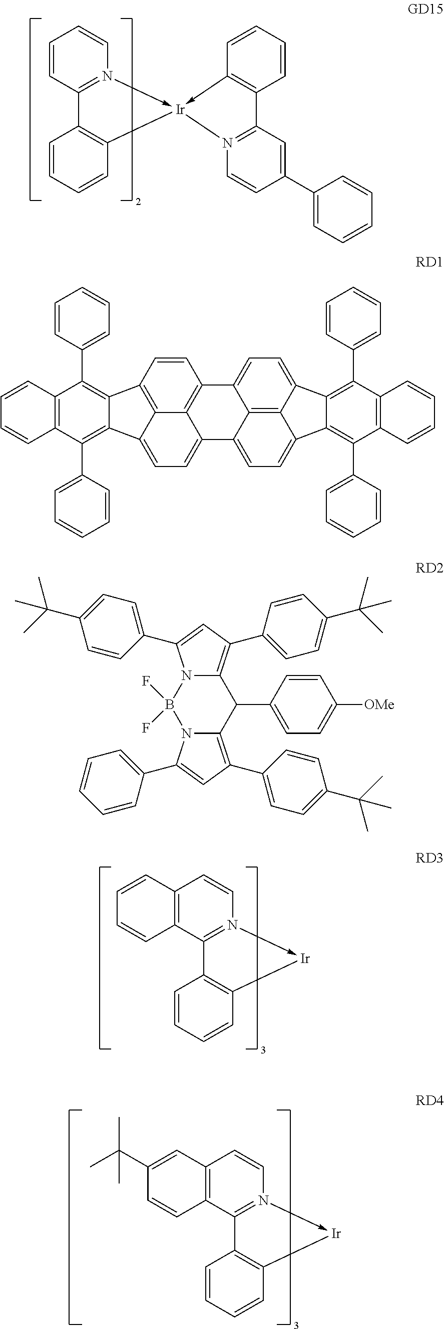

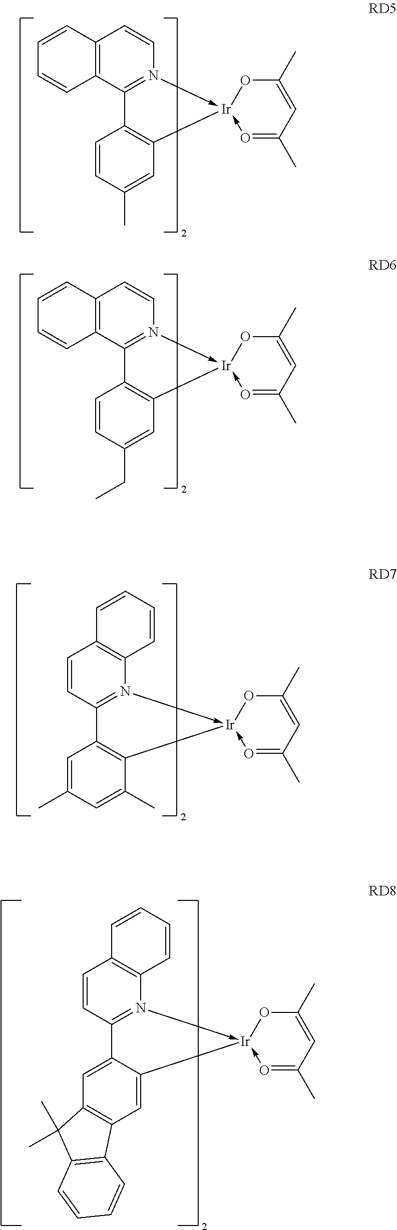

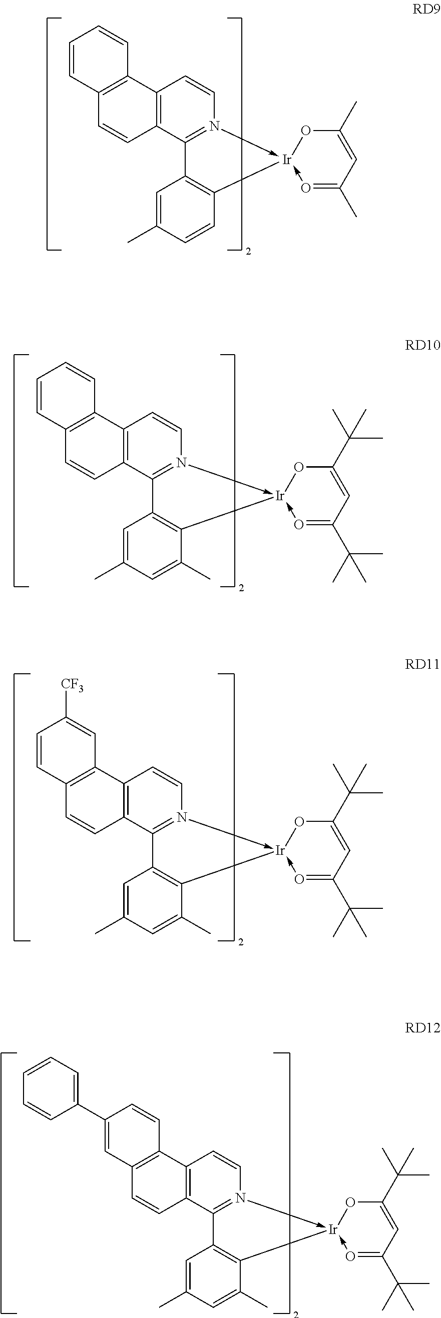

As a host or light emission assist material contained in the light-emitting layer, besides the organic compound according to an embodiment of the present disclosure, another host or light emission assist material can also be added. Examples of the other host or light emission assist material include, in addition to aromatic hydrocarbon compounds and derivatives thereof, carbazole derivatives, dibenzofuran derivatives, dibenzothiophene derivatives, triazine derivatives, organoaluminum complexes such as tris(8-quinolinolato)aluminum, organoberyllium complexes, polymers such as polyphenylene derivatives, polyphenylene vinylene derivatives, polyfluorene derivatives, and polyvinyl carbazole derivatives, copolymers thereof, and mixtures thereof.

Specific examples of the compound used as the host or light emission assist material contained in the light-emitting layer are shown below, but of course, the host or the light emission assist material is not limited thereto.

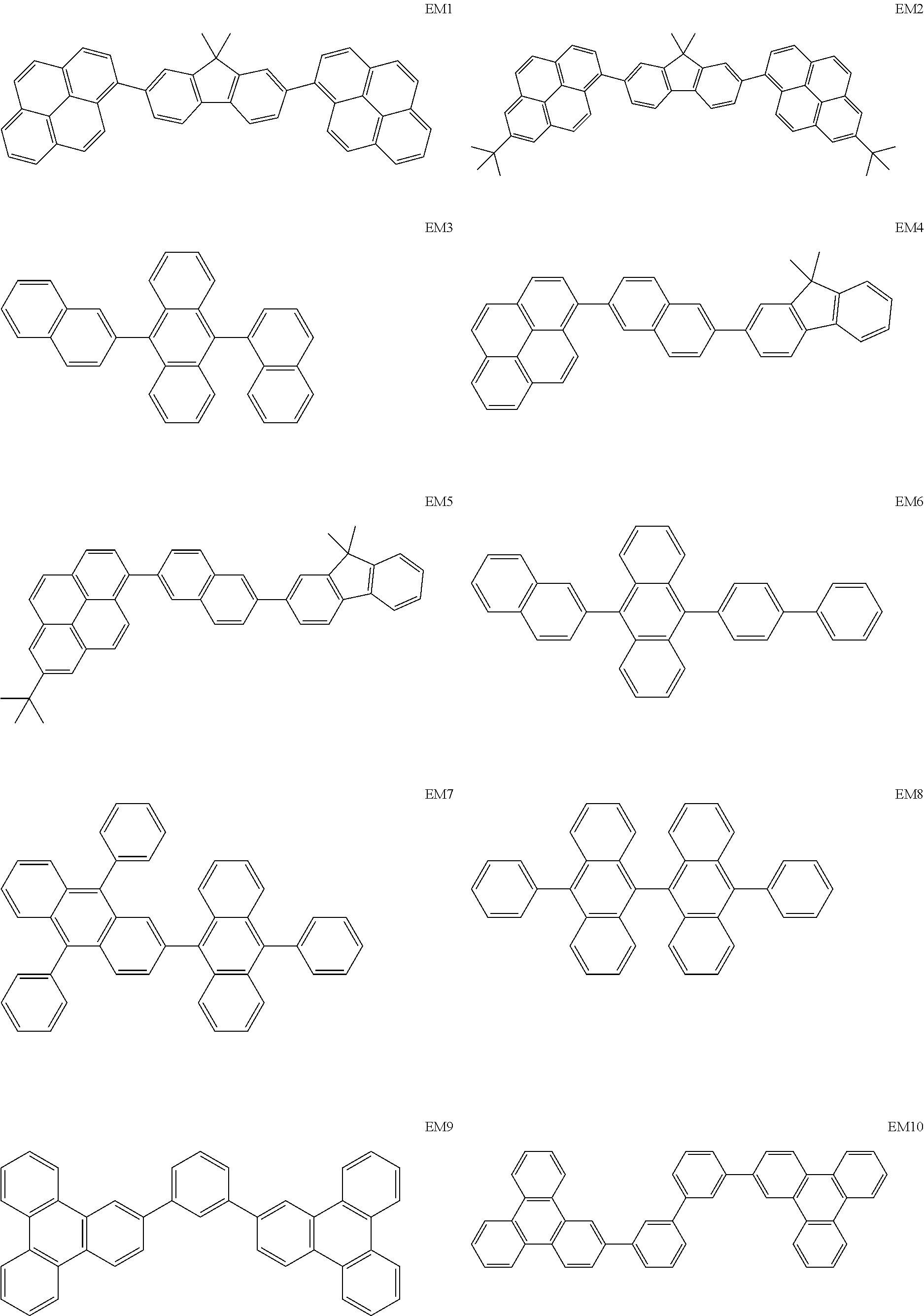

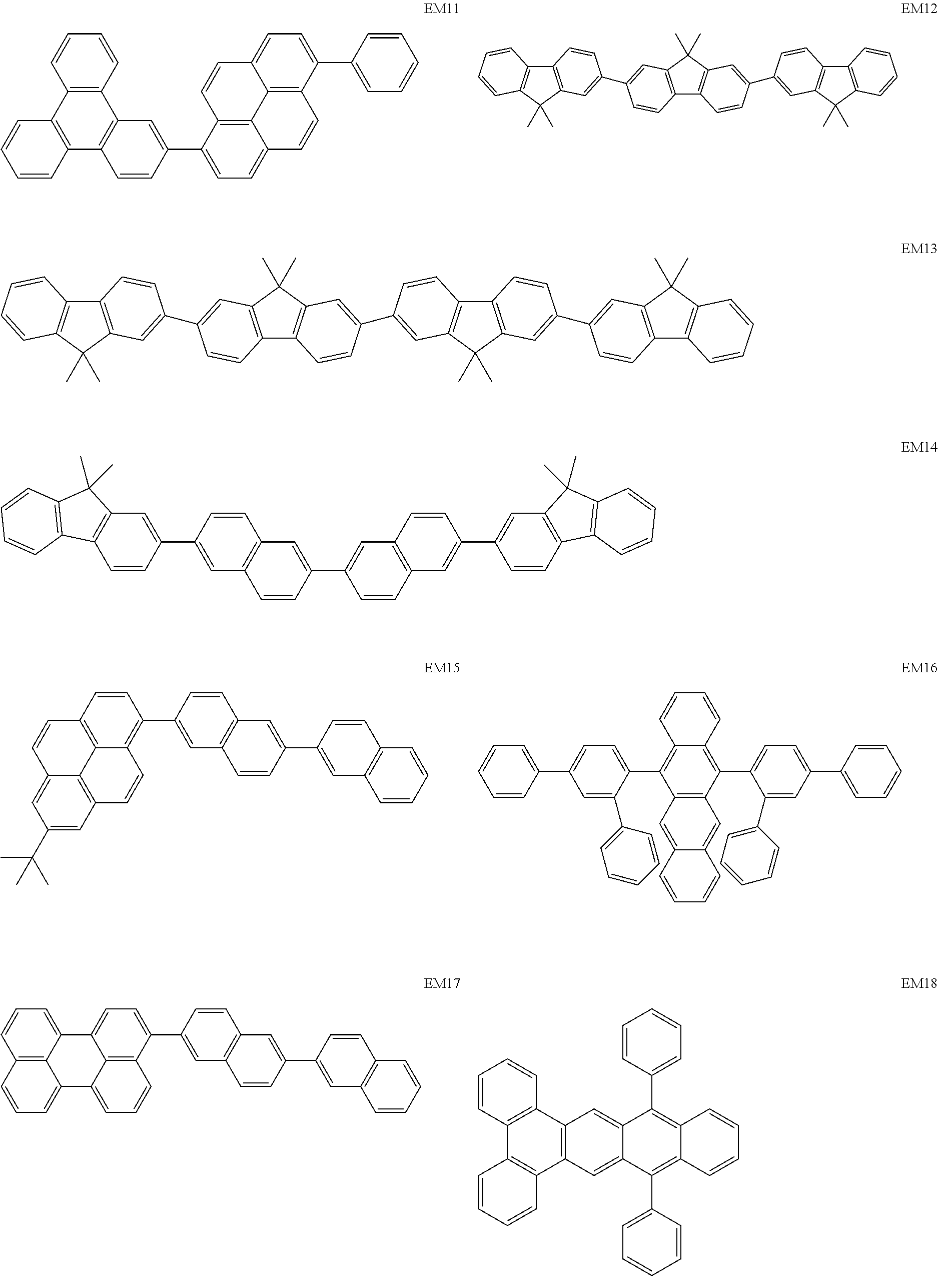

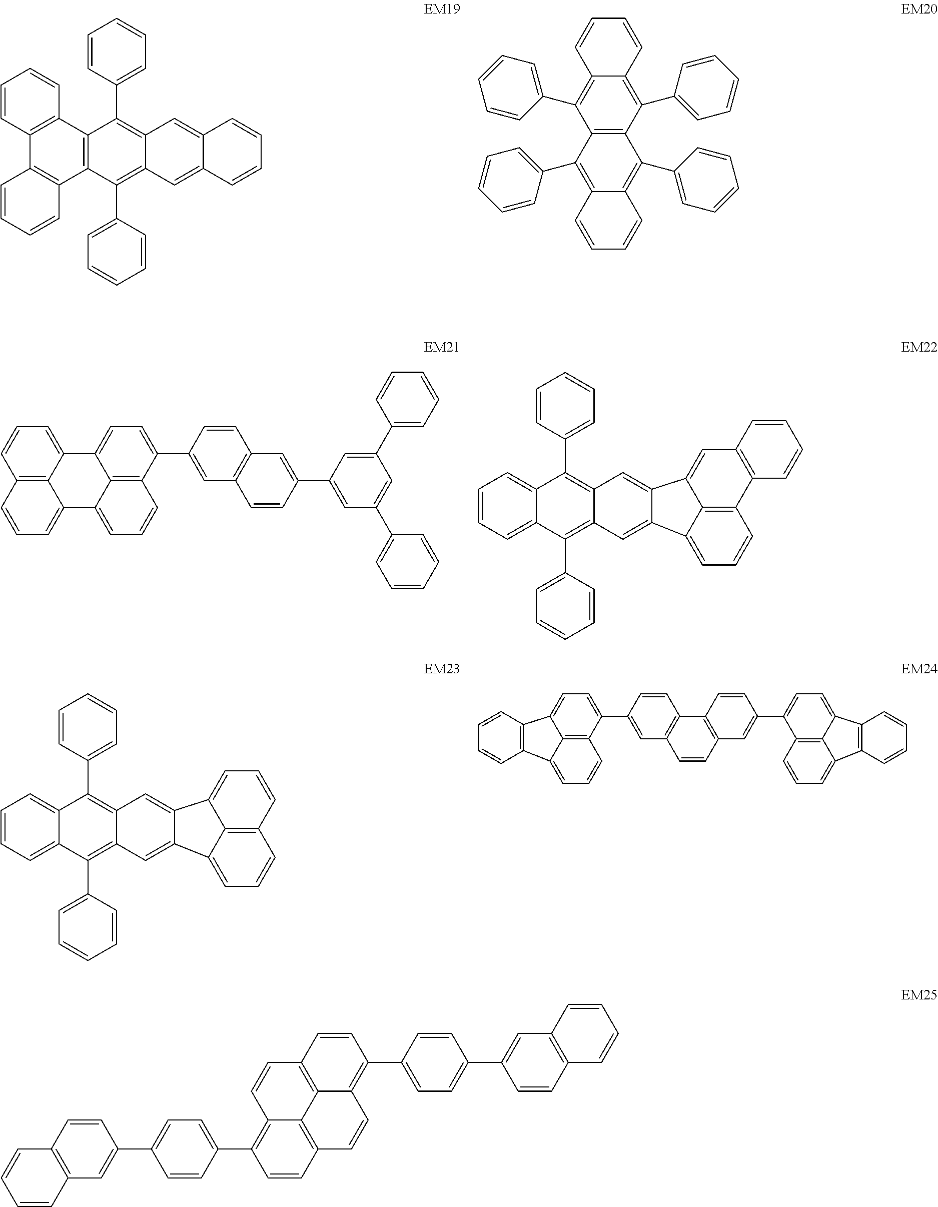

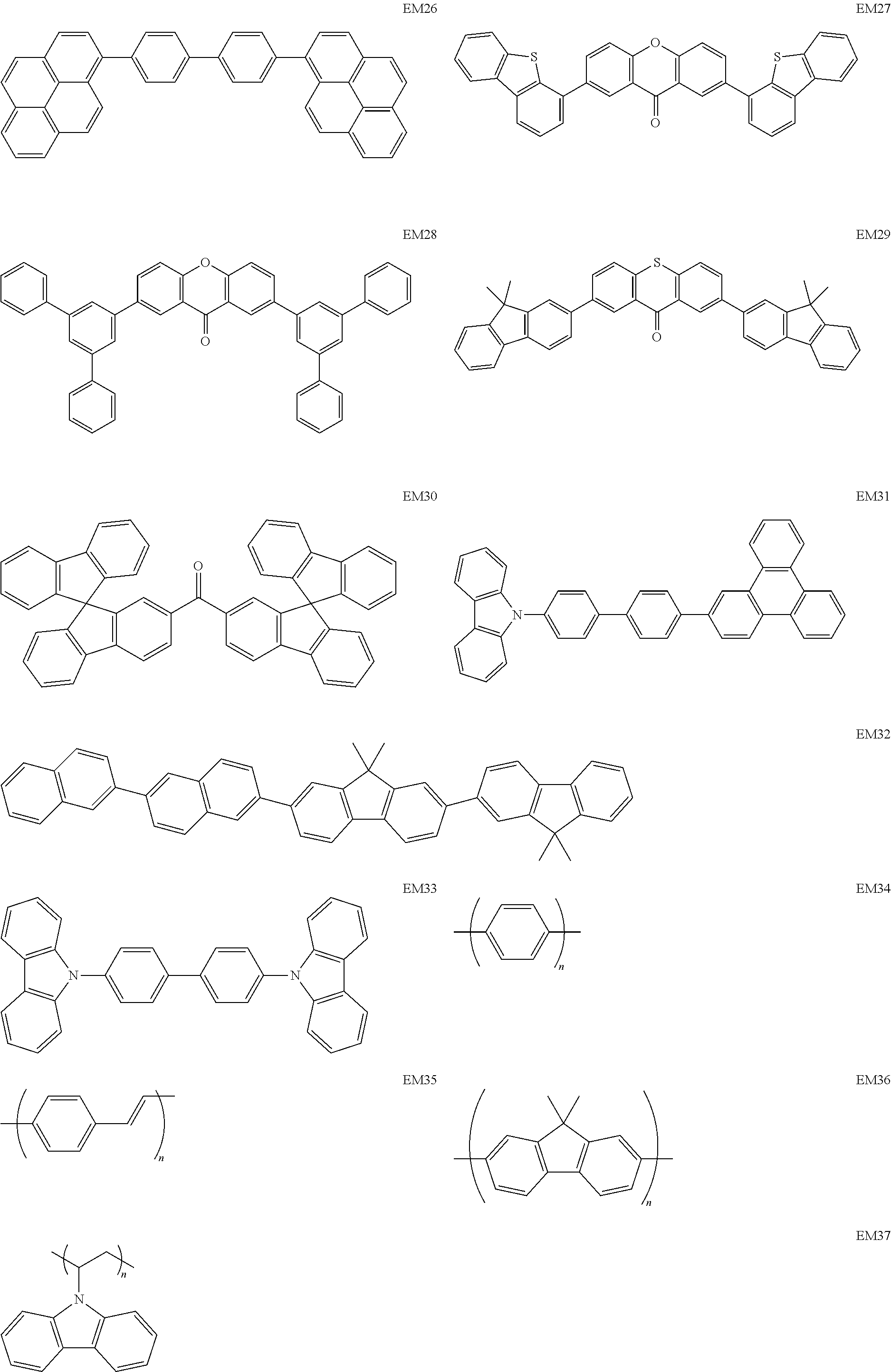

The electron transport material can be freely selected from materials capable of transporting electrons injected from the cathode to the light-emitting layer and is selected in consideration of, for example, the balance with the hole mobility of the hole transport material. Examples of the material having such electron transport performance include oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds (such as fluorene derivatives, naphthalene derivatives, chrysene derivatives, and anthracene derivatives). The above electron transport materials are also suitable for use in a hole-blocking layer.

Specific examples of the compound used as the electron transport material are shown below, but of course, the electron transport material is not limited thereto.

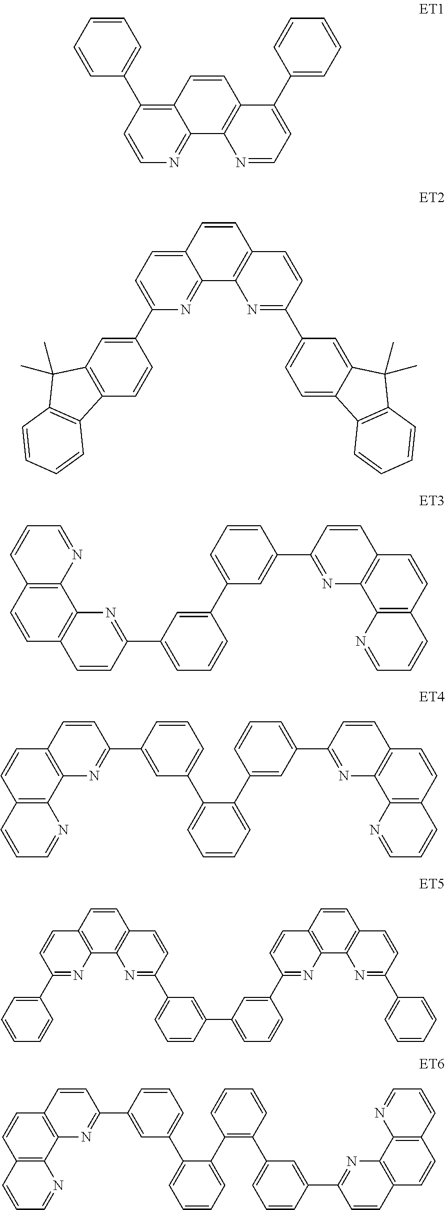

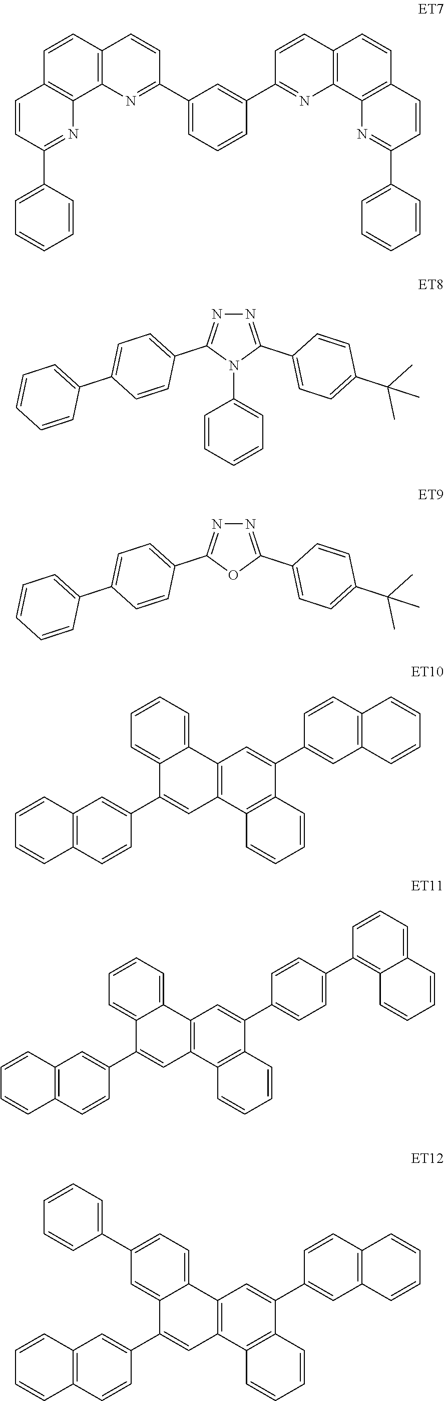

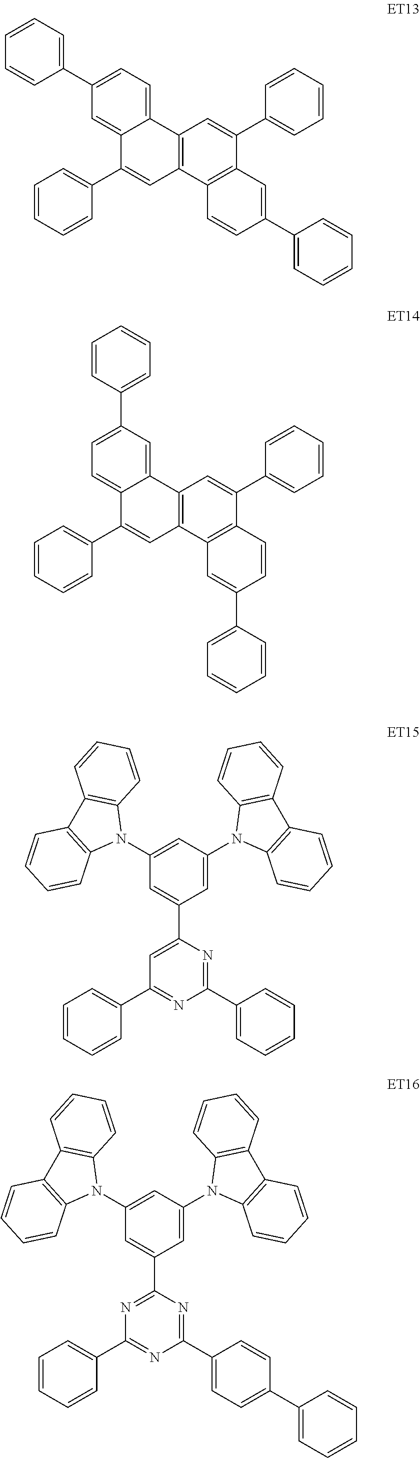

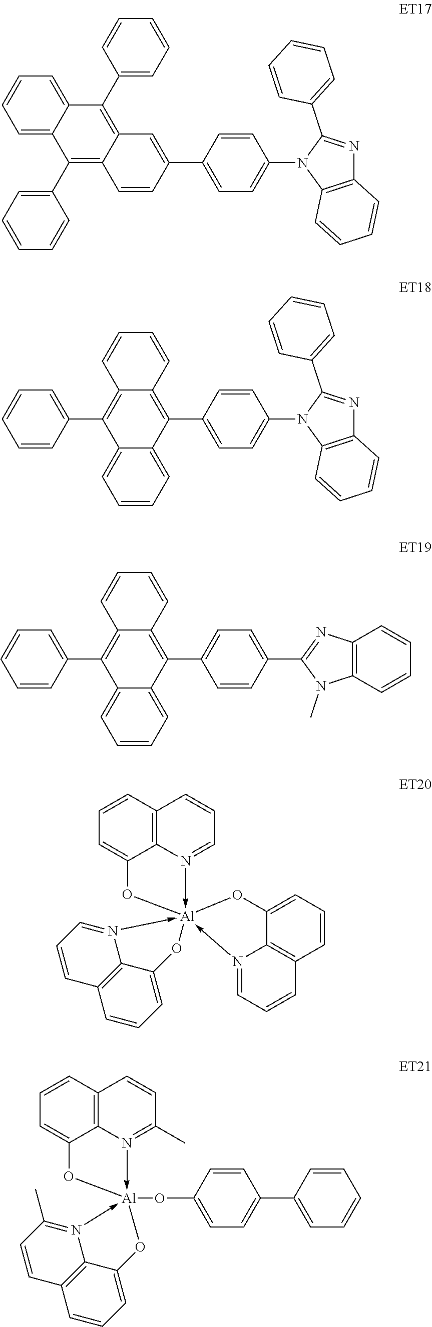

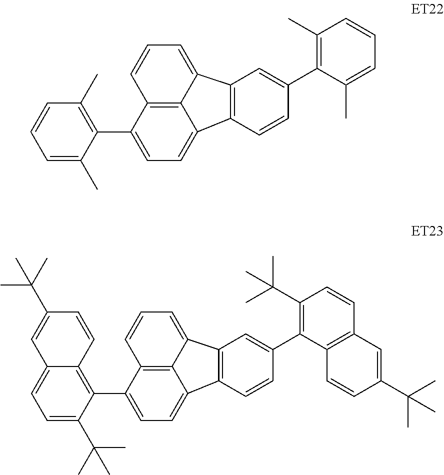

The electron injection material can be freely selected from materials capable of easily injecting electrons from the cathode and is selected in consideration of, for example, the balance with the hole injection property. As the organic compound, n-type dopants and reducing dopants are also included. Examples thereof include alkali metal-containing compounds such as lithium fluoride, lithium complexes such as lithium quinolinol, benzimidazolidene derivatives, imidazolidene derivatives, fulvalene derivatives, and acridine derivatives.

### Structure of Organic Light-Emitting Device

An organic light-emitting device is provided by forming a first electrode, an organic compound layer, and a second electrode on an insulating layer disposed on a substrate. A protective layer, a color filter, and the like may be disposed on the second electrode.

When a color filter is provided, a planarization layer may be provided between the color filter and the protective layer. The planarization layer can be formed of, for example, an acrylic resin. One of the first electrode and the second electrode may be an anode, and the other may be a cathode.

### Substrate

Examples of the substrate include quartz, glass, silicon wafers, resins, and metals. A switching element, such as a transistor, and wiring may be disposed on the substrate, and an insulating layer may be disposed thereon. The insulating layer may be formed of any material as long as a contact hole can be formed to ensure electrical connection between the anode and the wiring, and the insulating layer is insulated from wiring not to be connected. For example, a resin such as a polyimide, silicon oxide, or silicon nitride can be used.

### Electrodes

A pair of electrodes can be used for the electrodes. The pair of electrodes may be an anode and a cathode.

When an electric field is applied in a direction in which the organic light-emitting device emits light, an electrode with a high electric potential is the anode, and the other electrode is the cathode. In other words, the electrode that supplies holes to the light-emitting layer is the anode, and the electrode that supplies electrons to the light-emitting layer is the cathode.

The constituent material of the anode preferably has a work function as high as possible. Examples of the material that can be used include elemental metals such as gold, platinum, silver, copper, nickel, palladium, cobalt, selenium, vanadium, and tungsten; mixtures thereof, alloys of combinations thereof; and metal oxides such as tin oxide, zinc oxide, indium oxide, indium tin oxide (ITO), and indium zinc oxide. Electrically conductive polymers such as polyaniline, polypyrrole, and polythiophene can also be used.

These electrode substances may be used alone or in combination of two or more thereof. The anode may be composed of a single layer or a plurality of layers.

When the anode is used as a reflective electrode, for example, chromium, aluminum, silver, titanium, tungsten, or molybdenum, an alloy thereof, or a laminate thereof can be used. When the anode is used as a transparent electrode, a transparent conductive oxide layer made of, for example, indium tin oxide (ITO) or indium zinc oxide can be used; however, the anode is not limited thereto.

The electrode can be formed by photolithography.

On the other hand, the constituent material of the cathode is preferably a material having a low work function. Examples of the material include alkali metals such as lithium; alkaline earth metals such as calcium; other elemental metals such as aluminum, titanium, manganese, silver, lead, and chromium; and mixtures thereof. Alternatively, alloys of combinations of these elemental metals can also be used. For example, magnesium-silver, aluminum-lithium, aluminum-magnesium, silver-copper, and zinc-silver can be used. Metal oxides such as indium tin oxide (ITO) can also be used. These electrode substances may be used alone or in combination of two or more thereof. The cathode may be composed of a single layer or a plurality of layers. In particular, silver is preferably used, and a silver alloy is more preferably used to suppress aggregation of silver. The alloy may have any ratio as long as aggregation of silver can be suppressed. For example, the ratio may be 1:1.

The cathode may be a conductive oxide layer made of indium tin oxide (ITO) or the like to provide a top-emission device, or the cathode may be a reflective electrode made of aluminum (Al) or the like to provide a bottom-emission device. The cathode is not particularly limited. The method of forming the cathode is not particularly limited, and the use of direct-current or alternating-current sputtering or the like is more preferred because good film coverage is achieved and the resistance tends to decrease.

### Protective Layer

A protective layer may be disposed on the cathode. For example, a glass sheet provided with a moisture absorbent can be bonded to the cathode to reduce the entry of water or the like into the organic compound layer, thereby reducing the occurrence of display defects. In another embodiment, a passivation film made of silicon nitride or the like may be provided on the cathode to reduce the entry of water or the like into the organic compound layer. For example, after the formation of the cathode, the resulting substrate may be transferred to another chamber without breaking the vacuum, and a silicon nitride film having a thickness of 2 μm may be formed thereon as a protective layer by a CVD method. After the film formation by the CVD method, an atomic layer deposition method (ALD method) may be performed to form a protective layer.

### Color Filter

A color filter may be disposed on the protective layer. For example, a color filter that takes into account the size of the organic light-emitting device may be provided on another substrate, and the substrate may be bonded to a substrate on which the organic light-emitting device is provided. Alternatively, a color filter may be patterned on the above-described protective layer by photolithography. The color filter may be formed of a polymer.

### Planarization Layer

A planarization layer may be disposed between the color filter and the protective layer. The planarization layer may be formed of an organic compound. The organic compound may have a low molecular weight or a high molecular weight, and preferably has a high molecular weight.

The planarization layer may be provided above and below the color filter, and the constituent materials thereof may be the same or different. Specific examples of the constituent materials include polyvinylcarbazole resins, polycarbonate resins, polyester resins, ABS resins, acrylic resins, polyimide resins, phenolic resins, epoxy resins, silicone resins, and urea resins.

### Opposite Substrate

An opposite substrate may be disposed on the planarization layer. The opposite substrate is disposed at a position opposite to the above-described substrate and thus is referred to as the opposite substrate. The constituent material of the opposite substrate may be the same as that of the above-described substrate. When the above-described substrate is a first substrate, the opposite substrate may be a second substrate.

### Formation of Organic Compound Layer

An organic compound layer (such as a hole injection layer, a hole transport layer, an electron-blocking layer, a light-emitting layer, a hole-blocking layer, an electron transport layer, or an electron injection layer) constituting the organic light-emitting device according to an embodiment of the present disclosure is formed by a method described below.

The organic compound layer constituting the organic light-emitting device according to an embodiment of the present disclosure can be formed by a dry process or a wet process without particular limitation. Examples of the dry process that can be used include a vacuum evaporation method, an ionized evaporation method, sputtering, and plasma. Examples of the wet process that can be used include known coating methods (such as a spin coating method, a casting method, a microgravure coating method, a gravure coating method, a bar coating method, a roll coating method, a wire bar coating method, a dip coating method, a spray coating method, a screen printing method, a flexographic printing method, an offset printing method, an ink jet printing method, a capillary coating method, and a nozzle coating method) using a solution prepared by dissolving the compound in an appropriate solvent. Of these, for example, a vacuum evaporation method, an ionized evaporation method, an ink jet printing method, and a nozzle coating method are suitable for manufacturing an organic light-emitting device having a large area.

The thickness of each of the layers in the organic light-emitting device is usually preferably 1 nm to 10 μm. In particular, the thickness of the light-emitting layer of the organic compound layer is preferably 10 nm to 100 nm to obtain effective light emission characteristics.

When the light-emitting layer of the organic compound layer is formed by a wet process, the composition of such a layer is dissolved in a solvent to prepare an ink. The viscosity of the ink may be adjusted in accordance with the type of printing method. When such an ink is used in a printing method, such as an ink jet printing method, in which a solution passes through an ejection device, the viscosity is preferably 1 to 20 mPa s at 25° C. in order to reduce clogging and flight deflection during ejection.

A solvent having a boiling point of 70° C. to 300° C. at 1 atm can be usually used for the ink. The amount of organic solvent is usually 10 to 100 parts by mass relative to 1 part by mass of a material constituting each organic compound layer.

Drying of the coating film obtained by the wet process can be appropriately selected in accordance with the type of each layer. Usually, the heating can be performed at 100° C. to 250° C., preferably 110° C. to 200° C. for 5 minutes to 60 minutes in an air atmosphere or an inert gas (such as nitrogen or argon) atmosphere. The heating may be performed under normal pressure (1 atm) or reduced pressure (100 Pa to 0.1 MPa). The conditions of temperature, pressure, and time in the drying step can be adjusted so as to remove the solvent in each layer.

In the case of forming a film by the coating method, it is also possible to form the film in combination with an appropriate binder resin. Examples of the binder resin include, but are not limited to, polyvinyl carbazole resins, polycarbonate resins, polyester resins, ABS resins, acrylic resins, polyimide resins, phenolic resins, epoxy resins, silicone resins, and urea resins.

These binder resins may be used alone as a homopolymer or a copolymer, or in combination as a mixture of two or more thereof. Furthermore, additives, such as a known plasticizer, oxidation inhibitor, and ultraviolet absorbent may be used in combination, as needed.

### Pixel Circuit

A light-emitting apparatus may include a pixel circuit connected to a light-emitting device. The pixel circuit may be an active matrix-type circuit that independently controls light emission of first and second light-emitting devices. The active matrix-type circuit may be a voltage programming or current programming circuit. A driving circuit has a pixel circuit for each pixel. The pixel circuit may have a light-emitting device, a transistor that controls the emission luminance of the light-emitting device, a transistor that controls the timing of light emission, a capacitor that holds the gate voltage of the transistor that controls the emission luminance, and a transistor for establishing the connection to a GND without a light-emitting device interposed therebetween.

The light-emitting apparatus has a display region and a peripheral region disposed around the display region. The display region includes a pixel circuit, and the peripheral region includes a display control circuit. The mobility of a transistor constituting the pixel circuit may be smaller than the mobility of a transistor constituting the display control circuit.

The slope of the current-voltage characteristics of a transistor constituting the pixel circuit may be smaller than the slope of the current-voltage characteristics of a transistor constituting the display control circuit. The slope of the current-voltage characteristics can be measured by the so-called Vg-Ig characteristics.

The transistor constituting the pixel circuit is a transistor connected to a light-emitting device, such as the first light-emitting device.

### Pixel

The organic light-emitting apparatus includes a plurality of pixels. Each of the pixels includes subpixels configured to emit light of a color different from the other colors. For example, the subpixels may have RGB emission colors.

The pixels each emit light in a region that is also called a pixel aperture. This region is the same as a first region.

The pixel aperture may be 15 μm or less and may be 5 μm or more. More specifically, the pixel aperture may be, for example, 11 μm, 9.5 μm, 7.4 μm, or 6.4 μm.

The distance between subpixels may be 10 μm or less. Specifically, the distance may be 8 μm, 7.4 μm, or 6.4 μm. The pixels may be arranged in a known form in plan view. For example, the stripe arrangement, the delta arrangement, the PenTile arrangement, or the Bayer arrangement may be used. The shape of each subpixel in plan view may be any known shape. Examples of the shape include quadrangles, such as rectangles and rhombi, and hexagons. Of course, figures that are not exact shapes but are close to rectangles are also included in the rectangles. The shape of the subpixel and the pixel array can be used in combination.

### Applications of Organic Light-Emitting Device According to Embodiment of Present Disclosure

An organic light-emitting device according to an embodiment of the present disclosure can be used as a constituent member of a display apparatus or a lighting apparatus. Other applications include, for example, an exposure light source of an electrophotographic image forming apparatus, a backlight of a liquid crystal display apparatus, and a light-emitting apparatus including a color filter on a white light source.

The display apparatus may be an image information processing apparatus that includes an image input unit to which image information is input from an area CCD, a linear CCD, a memory card, or the like, that includes an information processing unit configured to process the input information, and that displays an input image on a display unit.

The display unit of an imaging apparatus or an ink jet printer may have a touch panel function. The method for driving the touch panel function may be an infrared radiation method, an electrostatic capacitance method, a resistive film method, or an electromagnetic induction method, and is not particularly limited. The display apparatus may be used for a display unit of a multifunction printer.

Next, a display apparatus according to the present embodiment will be described with reference to the drawings.

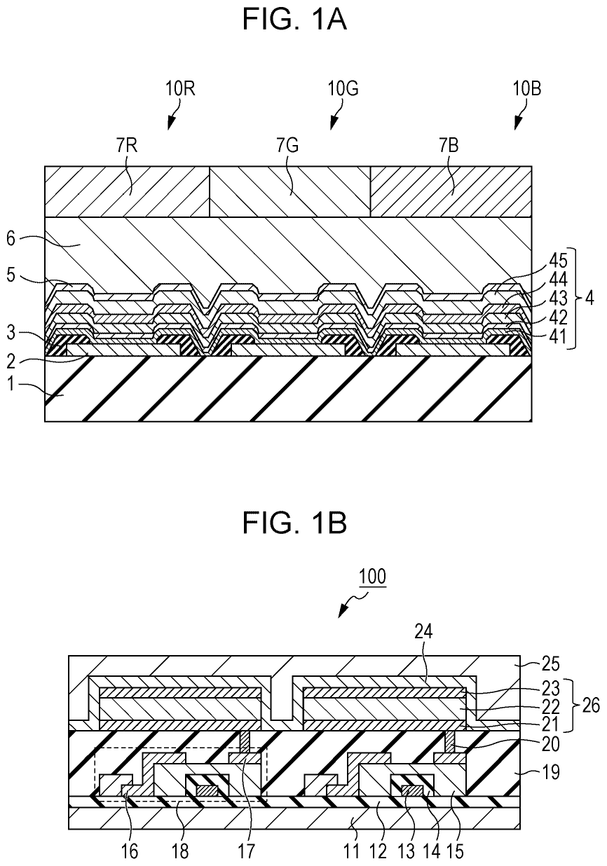

FIG. 1A is a schematic sectional view illustrating an example of a pixel constituting a display apparatus according to the present embodiment.

The pixel has subpixels 10. The subpixels are separated into 10R, 10G, and 10B according to their light emission. The emission color may be distinguished on the basis of the wavelength of light emitted from the light-emitting layer. Alternatively, light emitted from the subpixels may be selectively transmitted or color-converted through a color filter or the like. Each of the subpixels includes, on an interlayer insulating layer 1, a reflective electrode 2 which is a first electrode, an insulating layer 3 covering ends of the reflective electrode 2, an organic compound layer 4 covering the first electrode and the insulating layer, a transparent electrode 5, a protective layer 6, and a color filter 7.

The interlayer insulating layer 1 may have transistors and capacitor elements arranged in a layer disposed thereunder or an interior thereof.

Each transistor and the first electrode may be electrically connected to each other through a contact hole or the like not illustrated in the drawing.

The insulating layer 3 is also referred to as a bank or a pixel isolation film. The insulating layer 3 covers ends of the first electrode and is disposed so as to surround the first electrode. The portion where the insulating layer 3 is not disposed is in contact with the organic compound layer 4 and serves as a light-emitting region.

The organic compound layer 4 includes a hole injection layer 41, a hole transport layer 42, a first light-emitting layer 43, a second light-emitting layer 44, and an electron transport layer 45. The second electrode 5 may be a transparent electrode, a reflective electrode, or a semi-transparent electrode.

The protective layer 6 reduces the penetration of moisture into the organic compound layer. Although the protective layer is illustrated as a single layer in the drawing, the protective layer may be composed of a plurality of layers. Each layer may be an inorganic compound layer or an organic compound layer.

The color filter 7 is separated into 7R, 7G, and 7B according to the colors thereof. The color filter may be formed on a planarization film not illustrated in the drawing. Furthermore, a resin protective layer not illustrated in the drawing may be disposed on the color filter. The color filter may be formed on the protective layer 6 or may be provided on and then bonded to an opposite substrate such as a glass substrate.

FIG. 1B is a schematic sectional view illustrating an example of a display apparatus including organic light-emitting devices and transistors connected to the organic light-emitting devices. The transistors are each an example of an active element. The transistors may be thin-film transistors (TFTs).

A display apparatus 100 illustrated in FIG. 1B includes a substrate 11 made of glass, silicon, or the like and an insulating layer 12 provided on the substrate 11. On the insulating layer 12, active elements 18 such as TFTs are disposed, and a gate electrode 13, a gate insulating film 14, and a semiconductor layer 15 of each of the active elements are disposed. Each of the TFTs 18 is composed of the semiconductor layer 15, a drain electrode 16, and a source electrode 17. An insulating film 19 is disposed on the TFTs 18. An anode 21 constituting an organic light-emitting device 26 and the source electrode 17 are connected through a contact hole 20 provided in the insulating film 19.

The form of electrical connection between the electrodes (anode 21 and cathode 23) included in the organic light-emitting device 26 and the electrodes (source electrode 17 and drain electrode 16) included in the TFT is not limited to that illustrated in FIG. 1B. That is, any form may be employed as long as one of the anode and the cathode is electrically connected to one of the source electrode and the drain electrode of the TFT. The term “TFT” refers to a thin-film transistor.

In the display apparatus 100 illustrated in FIG. 1B, the organic compound layer is illustrated as a single layer. Alternatively, the organic compound layer 22 may be composed of a plurality of layers. A first protective layer 24 and a second protective layer 25 for reducing degradation of the organic light-emitting device 26 are disposed over the cathode 23.

Although transistors are used as the switching elements in the display apparatus 100 illustrated in FIG. 1B, other switching elements may be used instead of the transistors.

The transistors used in the display apparatus 100 illustrated in FIG. 1B are not limited to transistors that use a single-crystal silicon wafer and may also be thin-film transistors including an active layer on an insulating surface of a substrate. Examples of the material of the active layer include single-crystal silicon; non-single-crystal silicon such as amorphous silicon and microcrystalline silicon; and non-single-crystal oxide semiconductors such as indium zinc oxide and indium gallium zinc oxide. The thin-film transistors are also referred to as TFT elements.

The transistors included in the display apparatus 100 illustrated in FIG. 1B may be formed within a substrate, such as a Si substrate. Herein, the expression “formed within a substrate” means that transistors are produced by processing a substrate itself, such as a Si substrate. In other words, having transistors within a substrate can be considered to mean that a substrate and transistors are integrally formed.

In the organic light-emitting device according to the present embodiment, the emission luminance is controlled by the TFTs, which are one example of switching elements, and an image can be displayed at respective emission luminance levels by arranging a plurality of organic light-emitting devices in a plane. The switching elements according to the present embodiment are not limited to TFTs and may be transistors formed of low-temperature polysilicon or active-matrix drivers formed on a substrate, such as a Si substrate. The expression “on a substrate” can also be referred to as “within the substrate”. Whether transistors are formed within a substrate or TFTs are used is selected on the basis of the size of a display unit. For example, when the display unit has a size of about 0.5 inches, organic light-emitting devices are preferably disposed on a Si substrate.

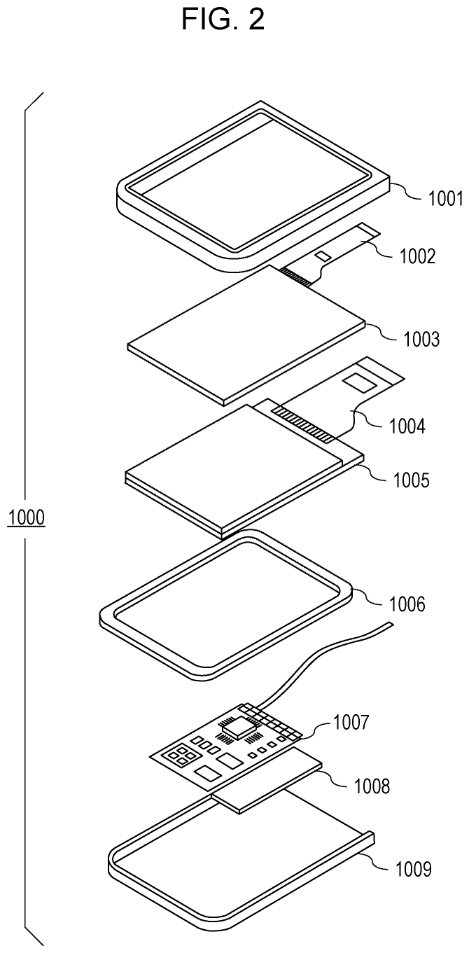

FIG. 2 is a schematic view illustrating an example of a display apparatus according to the present embodiment. A display apparatus 1000 may include an upper cover 1001 and a lower cover 1009, and a touch panel 1003, a display panel 1005, a frame 1006, a circuit substrate 1007, and a battery 1008 that are disposed between the upper cover 1001 and the lower cover 1009. The touch panel 1003 and the display panel 1005 are connected to flexible printed circuits FPC 1002 and 1004, respectively. Transistors are printed on the circuit substrate 1007. The battery 1008 is not necessarily installed unless the display apparatus is a mobile device or may be installed in another position even if the display apparatus is a mobile device.

The display apparatus according to the present embodiment may include a color filter having red, green, and blue portions. The red, green, and blue portions of the color filter may be arranged in a delta arrangement, a stripe arrangement, or a mosaic arrangement.

The display apparatus according to the present embodiment may be used for a display unit of a mobile terminal. In such a case, the display apparatus may have both a display function and an operation function. Examples of the mobile terminal include mobile phones such as smart phones, tablets, and head-mounted displays (HMDs).

The display apparatus according to the present embodiment may be used for a display unit of an imaging apparatus including an optical unit including a plurality of lenses and an imaging device configured to receive light that has passed through the optical unit. The imaging apparatus may include a display unit configured to display information acquired by the imaging device. The display unit may be a display unit exposed to the outside of the imaging apparatus or a display unit disposed in a viewfinder. The imaging apparatus may be a digital camera or a digital camcorder. The imaging apparatus can also be referred to as a photoelectric conversion apparatus.

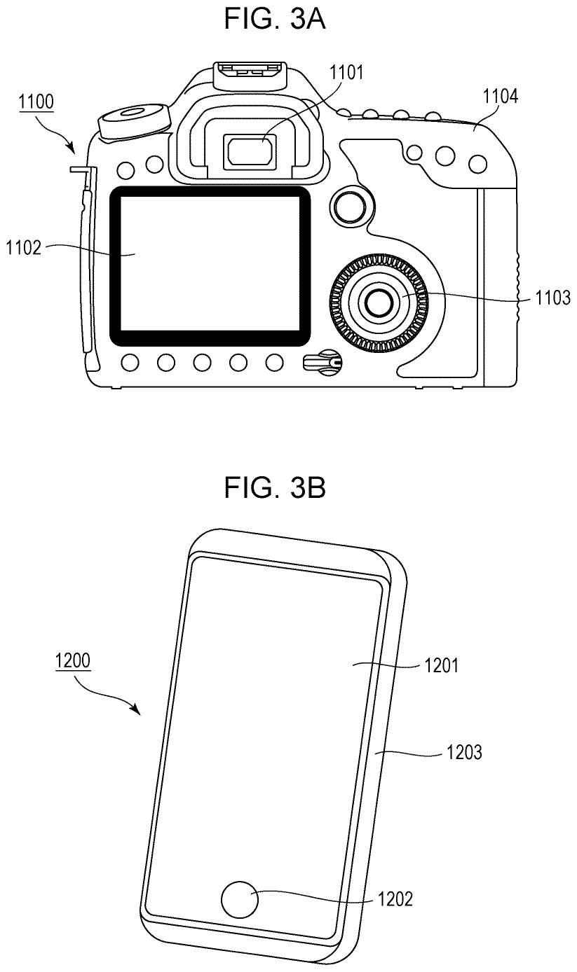

FIG. 3A is a schematic view illustrating an example of an imaging apparatus according to the present embodiment. An imaging apparatus 1100 may include a viewfinder 1101, a rear surface display 1102, an operation unit 1103, and a housing 1104. The viewfinder 1101 may include a display apparatus according to the present embodiment. In such a case, the display apparatus may display not only an image to be captured but also, for example, environmental information and imaging instructions. The environmental information may be, for example, the intensity of external light, the direction of external light, the moving speed of the photographic subject, and the possibility that the photographic subject may hide behind an obstacle.

Since the suitable timing for capturing an image is a very short period of time, it is desirable to display information as quickly as possible. Accordingly, a display apparatus that uses the organic light-emitting device of the present disclosure is preferably used. This is because organic light-emitting devices have a high response speed. Display apparatuses that use organic light-emitting devices can be more suitably used for such apparatuses required to have a high display speed than liquid crystal display apparatuses.

The imaging apparatus 1100 includes an optical unit not illustrated in the drawing. The optical unit includes a plurality of lenses and is configured to focus an image on an imaging device contained in the housing 1104. The focal point of the plurality of lenses can be adjusted by adjusting their relative positions. This operation can also be performed automatically.

FIG. 3B is a schematic view illustrating an example of electronic equipment according to the present embodiment. Electronic equipment 1200 includes a display unit 1201, an operation unit 1202, and a housing 1203. The housing 1203 may include therein circuits, a printed circuit board having the circuits, a battery, and a communication unit. The operation unit 1202 may be a button or a touch panel-type responsive unit. The operation unit 1202 may be a biometric recognition unit configured to, for example, recognize the fingerprints and release the lock. The electronic equipment that includes a communication unit can also be referred to as communication equipment. The electronic equipment 1200 may include a lens and an imaging device and thereby further have a camera function. An image captured by the camera function is displayed on the display unit 1201. Examples of the electronic equipment 1200 include smart phones and notebook computers.

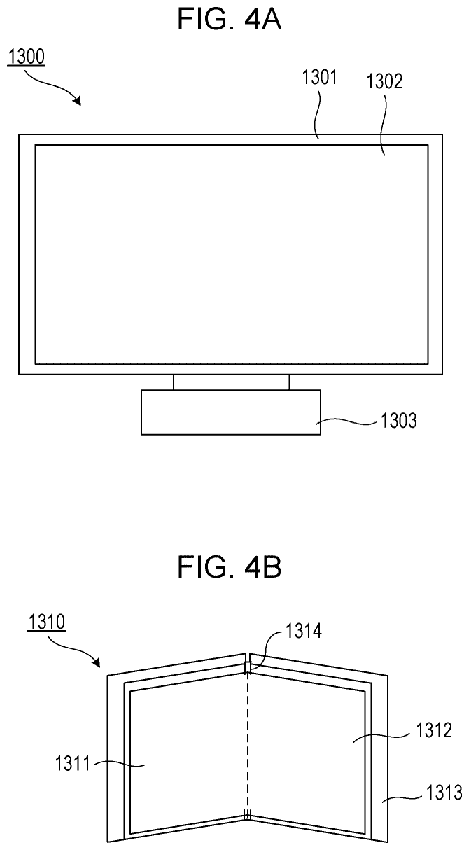

FIGS. 4A and 4B are schematic views each illustrating an example of a display apparatus according to the present embodiment. FIG. 4A illustrates a display apparatus such as a television monitor or a PC monitor. A display apparatus 1300 includes a frame 1301 and a display unit 1302. A light-emitting apparatus according to the present embodiment may be used as the display unit 1302.

The display apparatus 1300 includes a base 1303 that supports the frame 1301 and the display unit 1302. The base 1303 is not limited to the form illustrated in FIG. 4A. The lower side of the frame 1301 may also function as the base.

The frame 1301 and the display unit 1302 may be curved. The radius of curvature thereof may be 5,000 mm or more and 6,000 mm or less.

FIG. 4B is a schematic view illustrating another example of the display apparatus according to the present embodiment. A display apparatus 1310 illustrated in FIG. 4B is configured to be foldable and is a so-called foldable display apparatus. The display apparatus 1310 has a first display unit 1311, a second display unit 1312, a housing 1313, and a folding point 1314. The first display unit 1311 and the second display unit 1312 may include a light-emitting apparatus according to the present embodiment. The first display unit 1311 and the second display unit 1312 may be a single display apparatus without a joint. The first display unit 1311 and the second display unit 1312 can be separated at the folding point. The first display unit 1311 and the second display unit 1312 may respectively display different images or may together display a single image.

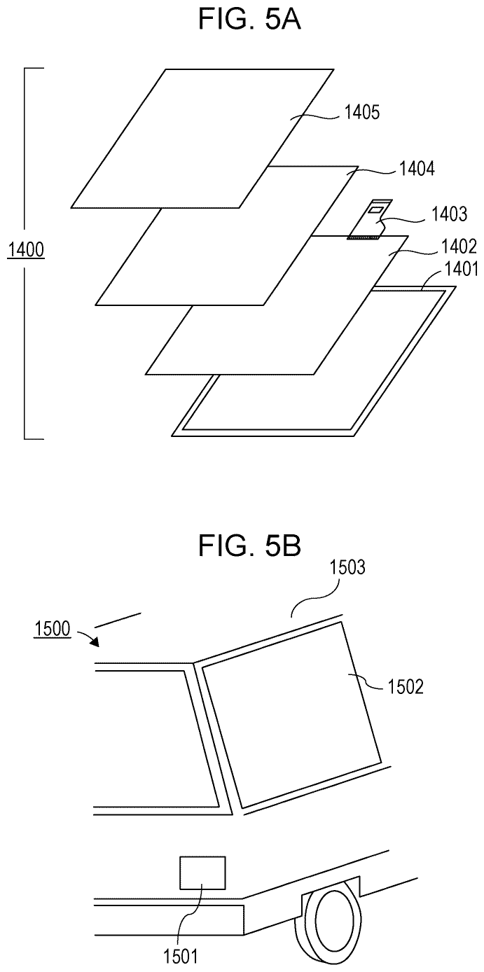

FIG. 5A is a schematic view illustrating an example of a lighting apparatus according to the present embodiment. A lighting apparatus 1400 may include a housing 1401, a light source 1402, a circuit substrate 1403, an optical film 1404, and a light diffusion unit 1405. The light source may include an organic light-emitting device according to the present embodiment. An optical filter may be a filter that improves the color rendering properties of the light source. The light diffusion unit can effectively diffuse light emitted from the light source and allow the light to reach a wide range, for example, for lighting up. The optical filter and the light diffusion unit may be disposed on the light-emitting side of the illumination. A cover may be optionally disposed on the outermost portion.

The lighting apparatus is, for example, an apparatus that illuminates the interior of a room. The lighting apparatus may emit light of a color such as white, natural white, or any other color from blue to red. The lighting apparatus may have a light modulation circuit configured to modulate the light.

The lighting apparatus may include an organic light-emitting device of the present disclosure and a power supply circuit connected to the organic light-emitting device. The power supply circuit is a circuit configured to convert an AC voltage into a DC voltage. The white is a color having a color temperature of 4,200 K, and the natural white is a color having a color temperature of 5,000 K. The lighting apparatus may include a color filter.

The lighting apparatus according to the present embodiment may include a heat dissipation unit. The heat dissipation unit dissipates heat in the apparatus to the outside of the apparatus and may be formed of, for example, a metal having a high specific heat or liquid silicone.

FIG. 5B is a schematic view of an automobile that is an example of a movable object according to the present embodiment. The automobile has a tail lamp that is an example of a lighting fixture. An automobile 1500 has a tail lamp 1501, and the tail lamp may light up when, for example, the brakes are applied.

The tail lamp 1501 may include an organic light-emitting device according to the present embodiment. The tail lamp 1501 may include a protective member that protects the organic light-emitting device. The protective member may be made of any material that has high strength to a certain extent and that is transparent, and is preferably made of polycarbonate or the like. The polycarbonate may be mixed with a furandicarboxylic acid derivative, an acrylonitrile derivative, or the like.

The automobile 1500 may include a car body 1503 and a window 1502 attached to the car body 1503. The window 1502 may be a transparent display unless it is a window for checking the front and rear of the automobile 1500. The transparent display may include an organic light-emitting device according to the present embodiment. In such a case, the constituent materials of the electrodes and the like of the organic light-emitting device are formed of transparent members.

The movable object according to the present embodiment may be, for example, a ship, an aircraft, or a drone. The movable object may include a body and a lighting fixture attached to the body. The lighting fixture may emit light to indicate the position of the body. The lighting fixture includes an organic light-emitting device according to the present embodiment.

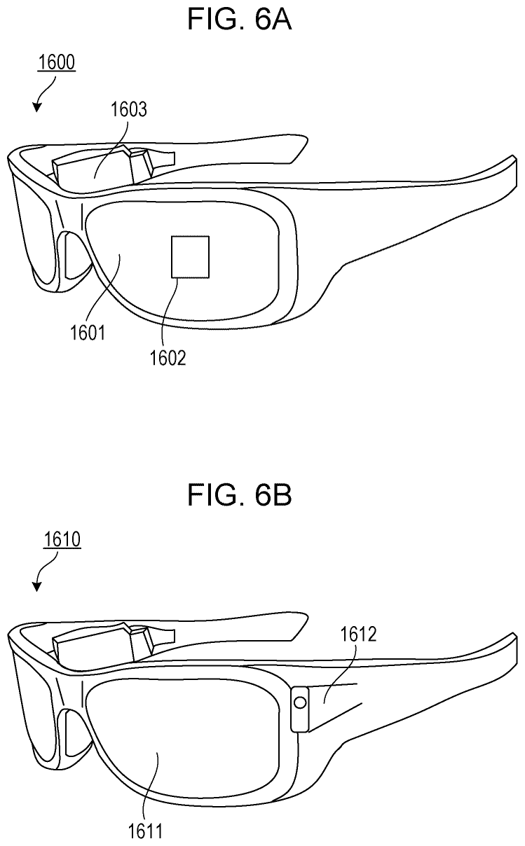

Application examples of the display apparatuses according to the above-described embodiments will be described with reference to FIGS. 6A and 6B. The display apparatuses are applicable to systems that can be worn as wearable devices, such as smart glasses, HMDs, and smart contact lenses. An imaging display apparatus used in such an application example includes an imaging apparatus that can photoelectrically convert visible light and a display apparatus that can emit visible light.

Glasses 1600 (smart glasses) according to an application example will be described with reference to FIG. 6A. An imaging apparatus 1602 such as a complementary metal-oxide semiconductor (CMOS) sensor or a single-photon avalanche diode (SPAD) is disposed on the front side of a lens 1601 of the glasses 1600. A display apparatus according to any of the embodiments described above is disposed on the back side of the lens 1601.

The glasses 1600 further include a control unit 1603. The control unit 1603 functions as a power supply that supplies electric power to the imaging apparatus 1602 and the display apparatus according to any of the embodiments. The control unit 1603 controls the operation of the imaging apparatus 1602 and the display apparatus. The lens 1601 includes an optical system for focusing light on the imaging apparatus 1602.

Glasses 1610 (smart glasses) according to an application example will be described with reference to FIG. 6B. The glasses 1610 have a control unit 1612, and the control unit 1612 includes an imaging apparatus corresponding to the imaging apparatus 1602 and a display apparatus. A lens 1611 includes an optical system for projecting light emitted from the imaging apparatus and the display apparatus in the control unit 1612, and an image is projected onto the lens 1611. The control unit 1612 functions as a power supply that supplies electric power to the imaging apparatus and the display apparatus and controls the operation of the imaging apparatus and the display apparatus. The control unit may have a gaze detection unit that detects the gaze of the wearer. Infrared rays may be used to detect the gaze. An infrared light-emitting unit emits infrared light toward an eyeball of the user who is gazing at a displayed image. Reflection of the infrared light from the eyeball is detected by an imaging unit including a light-receiving element to capture an image of the eyeball. A reducing unit configured to reduce light from the infrared light-emitting unit to a display unit in plan view is provided to reduce degradation of the image quality.

The gaze of the user for the displayed image is detected from the image of the eyeball captured with the infrared light. Any known method is applicable to the gaze detection using the captured image of the eyeball. As one example, a gaze detection method based on the Purkinje image formed by reflection of irradiation light on the cornea can be employed.

More specifically, a gaze detection process based on a pupil-corneal reflection method is performed. The gaze of the user is detected by calculating a gaze vector that indicates the direction (rotation angle) of the eyeball on the basis of the image of the pupil and the Purkinje image included in the captured image of the eyeball using the pupil-corneal reflection method.

The display apparatus according to an embodiment of the present disclosure may include an imaging apparatus including a light-receiving element and may control a displayed image of the display apparatus on the basis of gaze information of the user from the imaging apparatus.

Specifically, in the display apparatus, a first field-of-view region at which the user gazes and a second field-of-view region other than the first field-of-view region are determined on the basis of the gaze information. The first field-of-view region and the second field-of-view region may be determined by the control unit of the display apparatus or may be determined by receiving those determined by an external control unit.

In the display region of the display apparatus, the display resolution of the first field-of-view region may be controlled to be higher than the display resolution of the second field-of-view region. In other words, the resolution of the second field-of-view region may be lower than that of the first field-of-view region.

The display region includes a first display region and a second display region different from the first display region. A region with higher priority is determined from the first display region and the second display region on the basis of the gaze information. The first display region and the second display region may be determined by the control unit of the display apparatus or may be determined by receiving those determined by an external control unit. The resolution of the region with higher priority may be controlled to be higher than the resolution of the region other than the region with higher priority. In other words, the resolution of a region with relatively low priority may be low.

Artificial intelligence (AI) may be used to determine the first display region or the region with higher priority. The AI may be a model configured to estimate the angle of the gaze and the distance to a target object at the end of the gaze from the image of the eyeball by using, as teaching data, the image of the eyeball and the direction in which the eyeball in the image was actually gazing. The AI program may be stored in the display apparatus, the imaging apparatus, or an external apparatus. When the AI program is stored in an external apparatus, the AI program is transmitted through communication to the display apparatus.

In the case of controlling the display on the basis of visual recognition detection, the display apparatus according to an embodiment of the present disclosure can be preferably applied to smart glasses further including an imaging apparatus that captures an external image. The smart glasses can display the captured external information in real time.

As described above, the use of an apparatus that uses an organic light-emitting device according to the present embodiment enables a stable display for a long time with good image quality.

### EXAMPLES

Examples will be described below. However, the present disclosure is not limited to these Examples.

### Synthesis Example 1

### Synthesis of Compound (122)

Compound (122) was synthesized by the following procedure. First, the synthesis of an intermediate will be described.

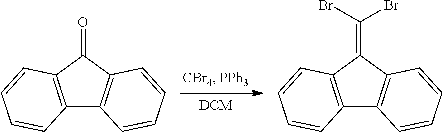 [SMILES: O=C1c2ccccc2-c2ccccc21 ; BrC(Br)=C1c2ccccc2-c2ccccc21]

In a nitrogen atmosphere, 6.70 g (20.0 mmol) of carbon tetrabromide, 10.7 g (40.0 mmol) of triphenylphosphine, and 100 mL of dichloromethane were added to a 200 mL recovery flask, and the mixture was stirred at room temperature. To the mixture, 1.84 g (10.0 mmol) of 9-fluorenone dissolved in 10 mL of dichloromethane was slowly added dropwise, and stirring was performed at room temperature for five hours.

After the reaction, 300 mL of heptane and Celite were added to the reaction mixture, stirring was performed, and the resulting reaction mixture was subjected to suction filtration. The filtrate was concentrated using a rotary evaporator and purified by silica gel column chromatography (mobile phase: heptane) to obtain 2.8 g of an intermediate. The structure was identified by MS.

Next, the synthesis of compound (122) will be described.

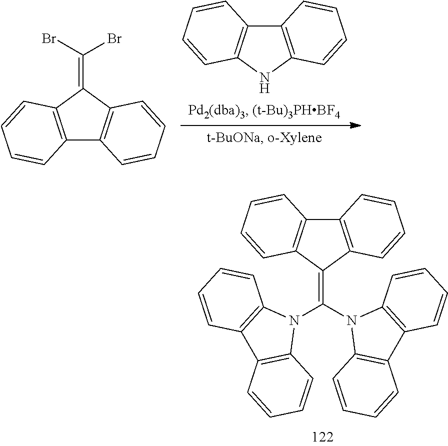 [SMILES: BrC(Br)=C1c2ccccc2-c2ccccc21 ; c1ccc2c(c1)[nH]c1ccccc12 ; c1ccc2c(c1)C(=C(n1c3ccccc3c3ccccc31)n1c3ccccc3c3ccccc31)c1ccccc1-2]

In a nitrogen atmosphere, 2.35 g (7.0 mmol) of the intermediate, 2.63 g (15.4 mmol) of carbazole, 2.06 g (21.0 mmol) of sodium tert-butoxide, 256.4 mg (0.21 mmol) of tris(dibenzylideneacetone)dipalladium, 310.9 mg (1.05 mmol) of tri(tert-butyl)phosphonium tetrafluoroborate, and 70 mL of o-xylene were added to a 200 mL three-necked flask and caused to react at 140° C. for 10 hours. After the reaction, a 1M aqueous ammonium chloride solution and ethyl acetate were added for liquid separation. Magnesium sulfate was added to the organic layer, and the organic layer was then subjected to suction filtration. The filtrate was concentrated using a rotary evaporator and purified by silica gel column chromatography (mobile phase: 5% ethyl acetate/heptane). The purified product was washed with methanol and dried, to obtain 1.50 g of a target product, i.e., compound (122). The structure was identified by MS.

### Example 1

Compound (122) prepared in Synthesis Example 1 was purified by sublimation at 360° C. and 3×10⁻³ Pa. The HPLC purity of the sublimate was 99.9%. The sublimate was used to produce, on a substrate, an organic light-emitting device having a structure of anode/hole injection layer/hole transport layer/electron-blocking layer/light-emitting layer/hole-blocking layer/electron transport layer/cathode that were disposed in that order, as described below.

An ITO film having a thickness of 100 nm was formed as an anode on a glass substrate by a sputtering method, and the resulting substrate was used as a transparent conductive supporting substrate (ITO substrate). Organic compound layers and electrode layers described below were successively formed on the ITO substrate by vapor deposition using resistance heating in a vacuum chamber at 1×10⁻⁵ Pa. In this process, the organic light-emitting device was produced such that the area of the opposing electrodes was 3 mm².
Hole injection layer (10 nm) HT16
Hole transport layer (40 nm) HT1
Light-emitting layer (30 nm) host: compound (122) (96% by weight), dopant: RD11 (4% by weight)
Electron transport layer (30 nm) ET20
Metal electrode layer 1 (15 nm) LiF
Metal electrode layer 2 (100 nm) Al

Next, the resulting structure was covered with a protective glass plate and sealed with an acrylic resin adhesive in a dry air atmosphere so that degradation of the organic light-emitting device due to moisture adsorption did not occur.

### Examples 2 to 36

Compounds (101) to (136) were purified by sublimation in the same manner as in the case of compound (122) of Example 1 to obtain sublimates having an HPLC purity of 99.9%. Organic light-emitting devices were produced in the same manner as in Example 1 except that the above sublimates were used instead of the sublimate of Example 1.

### Comparative Example 1

Compound A was purified by sublimation in the same manner as in the case of compound (122) of Example 1 to obtain a sublimate having an HPLC purity of 99.9%. An organic light-emitting device was produced in the same manner as in Example 1 except that the sublimate of this Comparative Example 1 was used instead of the sublimate of Example 1.

### Comparative Example 2

Compound B was purified by sublimation in the same manner as in the case of compound (122) of Example 1 to obtain a sublimate having an HPLC purity of 99.9%. An organic light-emitting device was produced in the same manner as in Example 1 except that the sublimate of this Comparative Example 2 was used instead of the sublimate of Example 1.

### Comparative Example 3

Compound D was purified by sublimation in the same manner as in the case of compound (122) of Example 1 to obtain a sublimate having an HPLC purity of 99.9%. An organic light-emitting device was produced in the same manner as in Example 1 except that the sublimate of this Comparative Example 3 was used instead of the sublimate of Example 1.

### Reference Example 1

Compound C was purified by sublimation in the same manner as in the case of compound (122) of Example 1 to obtain a sublimate having an HPLC purity of 99.9%. An organic light-emitting device was produced in the same manner as in Example 1 except that the sublimate of this Reference Example 1 was used instead of the sublimate of Example 1.

### Evaluation of Organic Light-Emitting Device

The organic light-emitting devices produced using light-emitting compositions obtained as described above were evaluated for the following item.

The external quantum efficiency of each of the organic light-emitting devices at 10 mA/cm² was measured and evaluated as a relative value when the value of the external quantum efficiency of the organic light-emitting device produced in Reference Example 1 was set to 1.0. The evaluation was conducted in accordance with the following evaluation criteria.
A: The relative value was 2.2 or more.
B: The relative value was 2.0 or more and less than 2.2.
C: The relative value was less than 2.0.

In the present disclosure, “A” and “B” in the evaluation criteria in the item below are acceptable levels, and “C” is an unacceptable level. The evaluation results are shown in Table 1.

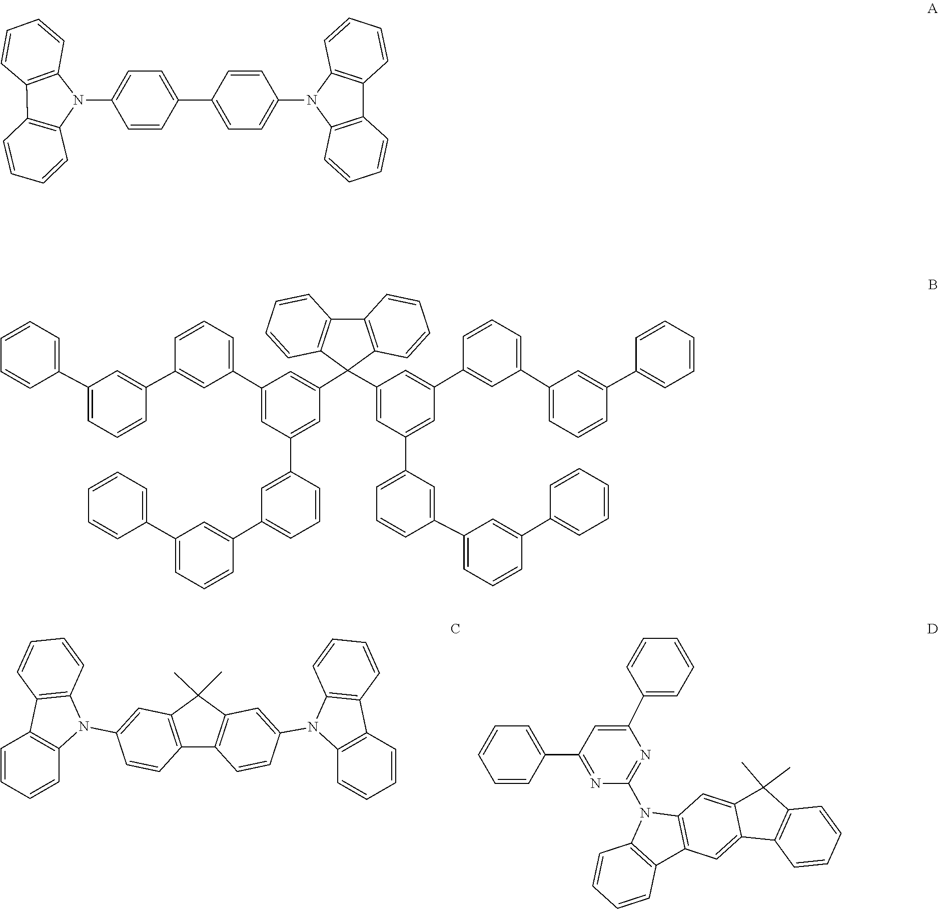

TABLE 1

| | | | EQE | |
|---|---|---|---|---|
| | | | relative | |
| Example | Compound | Dopant | value | Evaluation |
| 1 | 122 | RD11 | 2.30 | A |
| 2 | 101 | RD11 | 2.00 | B |
| 3 | 102 | RD11 | 2.02 | B |
| 4 | 103 | RD11 | 2.00 | B |
| 5 | 104 | RD11 | 2.09 | B |
| 6 | 105 | RD11 | 2.05 | B |
| 7 | 106 | RD11 | 2.07 | B |
| 8 | 107 | RD11 | 2.09 | B |
| 9 | 108 | RD11 | 2.02 | B |
| 10 | 109 | RD11 | 2.05 | B |
| 11 | 110 | RD11 | 2.00 | B |
| 12 | 111 | RD11 | 2.08 | B |
| 13 | 112 | RD11 | 2.09 | B |
| 14 | 113 | RD11 | 2.03 | B |
| 15 | 114 | RD11 | 2.10 | B |
| 16 | 115 | RD11 | 2.09 | B |
| 17 | 116 | RD11 | 2.20 | A |
| 18 | 117 | RD11 | 2.23 | A |
| 19 | 118 | RD11 | 2.27 | A |
| 20 | 119 | RD11 | 2.27 | A |
| 21 | 120 | RD11 | 2.23 | A |
| 22 | 121 | RD11 | 2.26 | A |
| 23 | 123 | RD11 | 2.37 | A |
| 24 | 124 | RD11 | 2.38 | A |
| 25 | 125 | RD11 | 2.17 | B |
| 26 | 126 | RD11 | 2.13 | B |
| 27 | 127 | RD11 | 2.18 | B |
| 28 | 128 | RD11 | 2.08 | B |
| 29 | 129 | RD11 | 2.18 | B |
| 30 | 130 | RD11 | 2.15 | B |
| 31 | 131 | RD11 | 2.27 | A |
| 32 | 132 | RD11 | 2.26 | A |
| 33 | 133 | RD11 | 2.25 | A |
| 34 | 134 | RD11 | 2.22 | A |
| 35 | 135 | RD11 | 2.20 | A |
| 36 | 136 | RD11 | 2.32 | A |
| Comparative Example 1 | Compound A | RD11 | 0.91 | C |
| Comparative Example 2 | Compound B | RD11 | 1.27 | C |
| Comparative Example 3 | Compound D | RD11 | 1.64 | C |
| Reference Example 1 | Compound C | RD11 | 1.00 | — |

As described above, the organic compound according to the present disclosure can constitute an organic light-emitting device having high light emission efficiency and high external quantum efficiency.

The present disclosure can provide an organic compound that exhibits high light emission efficiency when used in an organic light-emitting device.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-013573, filed Jan. 30, 2025, which is hereby incorporated by reference herein in its entirety.

What is claimed is:

1. An organic compound represented by formula (1) or formula (2): 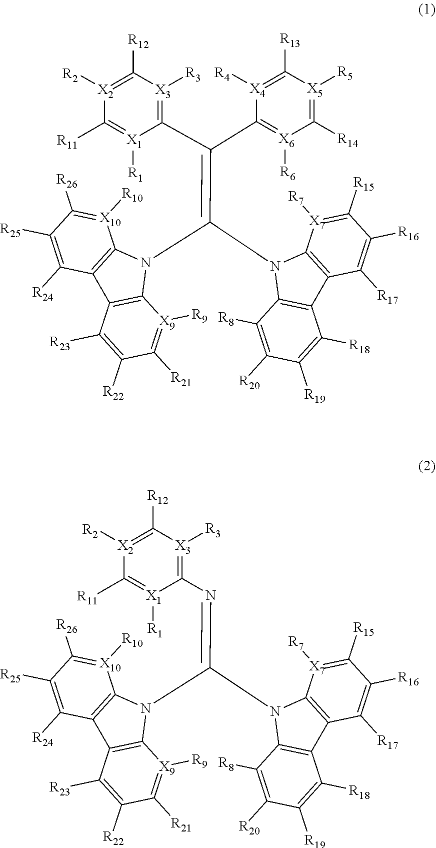
wherein in formula (1) and formula (2), X₁ to X₁₀ are each a carbon atom or a nitrogen atom,
R₁ to R₂₆ are each independently selected from the group consisting of a hydrogen atom, a deuterium atom, a halogen atom, a substituted or unsubstituted alkyl group, a substituted or unsubstituted cycloalkyl group, a substituted or unsubstituted aryl group, a substituted or unsubstituted heteroaryl group, a substituted or unsubstituted alkoxy group, a substituted or unsubstituted aryloxy group, a substituted or unsubstituted acyl group, a substituted or unsubstituted alkoxycarbonyl group, a substituted or unsubstituted amino group, a substituted or unsubstituted imino group, a substituted or unsubstituted silyl group, a substituted or unsubstituted sulfanyl group, a substituted or unsubstituted sulfinyl group, a substituted or unsubstituted sulfonyl group, and a cyano group, and
R₃ and R₄ are optionally bonded together to form a ring structure.

2. The organic compound according to claim 1, wherein X₁ to X₁₀ are each a carbon atom.

3. The organic compound according to claim 1, wherein R₁ to R₂₆ are any of a hydrogen atom, a deuterium atom, the alkyl group, the aryl group, the silyl group, and the cyano group.

4. The organic compound according to claim 1,
wherein in a case that R₃ and R₄ are bonded together to form a ring structure:
R₃ and R₄ are directly bonded to each other to form a five-membered ring, and,
R₁, R₂, and R₅ to R₂₆ are any of a hydrogen atom, a deuterium atom, the alkyl group, the aryl group, the heteroaryl group, the silyl group, and the cyano group, and
wherein in a case that R₃ and R₄ are not bonded together to form a ring structure, R₃ and R₄ form any of a methylene group, an ethylene group, an ether group, a thioether group, a sulfone group, a sulfoxide group, an imino group, and a carbonyl group that optionally have a substituent.

5. The organic compound according to claim 4, wherein R₃ and R₄ are directly bonded to each other to form a five-membered ring.

6. A light-emitting composition comprising:
the organic compound according to claim 1; and
a light-emitting organic compound.

7. An organic light-emitting device comprising:
a first electrode;
a second electrode; and
an organic compound layer disposed between the first electrode and the second electrode,
wherein the organic compound layer contains the organic compound according to claim 1.

8. The organic light-emitting device according to claim 7,
wherein the organic compound layer is a light-emitting layer,
the light-emitting layer further contains a first organic compound, and
a lowest excited triplet energy of the first organic compound is lower than a lowest excited triplet energy of the organic compound.

9. The organic light-emitting device according to claim 8,
wherein the light-emitting layer further contains a second organic compound, and
a lowest excited triplet energy of the second organic compound is lower than the lowest excited triplet energy of the organic compound.

10. The organic light-emitting device according to claim 9,
wherein the light-emitting layer further contains a third organic compound, and
a lowest excited triplet energy of the third organic compound is higher than the lowest excited triplet energy of the second organic compound.

11. The organic light-emitting device according to claim 9,
wherein the organic compound layer further includes a first charge transport layer disposed between the first electrode and the light-emitting layer and a second charge transport layer disposed between the second electrode and the light-emitting layer, and
the first electrode is in contact with the first charge transport layer, and the second electrode is in contact with the second charge transport layer.

12. The organic light-emitting device according to claim 11, wherein a lowest excited triplet energy of the first charge transport layer is higher than the lowest excited triplet energy of the first organic compound, and a lowest excited triplet energy of the second charge transport layer is higher than the lowest excited triplet energy of the first organic compound.

13. A display apparatus comprising:
a plurality of pixels,
wherein at least one of the plurality of pixels includes the organic light-emitting device according to claim 7 and a transistor connected to the organic light-emitting device.

14. An imaging apparatus comprising:
an optical unit including a plurality of lenses;
an imaging device configured to receive light that has passed through the optical unit; and
a display unit configured to display an image captured by the imaging device,
wherein the display unit includes the organic light-emitting device according to claim 7.

15. Electronic equipment comprising:
a display unit including the organic light-emitting device according to claim 7;
a housing provided with the display unit; and
a communication unit provided in the housing and configured to communicate with an external unit.

16. A lighting apparatus comprising:
a light source including the organic light-emitting device according to claim 7; and
a light diffusion unit or an optical film configured to transmit light emitted from the light source.

17. A movable object comprising:
a lighting fixture including the organic light-emitting device according to claim 7; and
a body provided with the lighting fixture.
